(19) United States
(12) Patent Application Publication
(10) Pub. No.: US 2024/0210386 A1
(43) Pub. Date: Jun. 27, 2024

(54) PURIFICATION OF HLA-SPECIFIC ANTIBODIES

(71) Applicant: One Lambda, Inc., West Hills, CA (US)

(72) Inventors: Rui Pei, West Hills, CA (US); Quansheng Zhu, North Hollywood, CA (US); Tri Vu, Pasadena, CA (US); David Philip Lowe, Alsager (GB)

(73) Assignee: One Lambda, Inc., West Hills, CA (US)

(21) Appl. No.: 18/537,521

(22) Filed: Dec. 12, 2023

Related U.S. Application Data
(60) Provisional application No. 63/432,237, filed on Dec. 13, 2022.
(60) Provisional application No. 63/543,974, filed on Oct. 13, 2023.

(51) Int. Cl. G01N 33/543; G01N 33/557; G01N 33/68

(52) U.S. Cl. CPC G01N 33/54326; G01N 33/557; G01N 33/6878

(57) ABSTRACT

Human leukocyte antigen (HLA) coated magnetic bead compositions, panels and methods of use thereof. A method of isolating HLA-specific antibodies including: contacting the composition of the disclosure with a liquid sample to specifically bind antibodies present in the liquid sample to HLA immobilized on magnetic beads; separating magnetic beads having antibodies bound to HLA of the magnetic beads from the liquid sample; and isolating the bound antibodies.

### CROSS REFERENCE TO RELATED APPLICATION(S)

This application claims the benefit of priority to U.S. Provisional Patent Application No. 63/432,237, filed Dec. 13, 2023, and U.S. Provisional Patent Application No. 63/543,974, filed Oct. 13, 2023, the disclosures of which are considered part of, and incorporated in their entireties by reference in the disclosure of this application.

### BACKGROUND

### Field

The present invention relates generally to immunology, and more particularly to human leukocyte antigen (HLA) magnetic bead compositions, panels and methods of use thereof to isolate HLA-specific antibodies.

### Background Information

One of the significant challenges in achieving successful long-term outcomes in solid organ transplantation is the presence of donor specific antibodies (DSA) that target human leukocyte antigens (HLA). The characterization of DSA was revolutionized in the early 2000's with development and commercial release of single antigen coated bead detection assays. However, the widespread adoption of these single antigen assays revealed the true nature and complexity of these antibodies, enabling further research into the mechanisms of interaction between these antibodies and their target antigens.

Exposure to foreign HLA, including exposure resulting from organ transplant, blood transfusion, or pregnancy, generally leads to proliferation of plasma cells and, in turn, production of antibodies by plasma cells to allogeneic HLA. DSA are associated with antibody-mediated rejection (AMR) in transplant patients which adversely impacts long-term survival.

Although a perfect or near-perfect match between donor and recipient HLA antigens is ideal, such matches are generally impossible due to the high degree of polymorphism in the HLA system. As a result, almost all recipients of organ transplants will be exposed to HLA mismatches.

HLA-specific antibodies recognize clusters of accessible polymorphic amino acid residues shared by target HLAs, termed functional epitopes or eplets. Some eplets are antibody-verified using alloantibodies and mouse monoclonal antibodies. Some eplets are theoretical, identified based on HLA single antigen binding reactivity, sequence alignment, and protein structure analysis. Different combinations of these eplets exist across various HLA variants, with a single HLA variant typically carrying numerous potential eplets.

Various tools have been developed for identifying and verifying eplets. An example is the HLA Matchmaker algorithm (see, Duquesnoy R J. Hum Immunol. 2002; 63(5):339-352. doi: 10.1016/s0198-8859(02)00382-8; Duquesnoy R J. Hum Immunol. 2006; 67(11):847-862. doi: 10.1016/j.humimm.2006.08.001; and Duquesnoy et al. Hum Immunol. 2007; 68(1): 12-25. doi: 10.1016/j.humimm.2006.10.003), which identifies eplets by analyzing patient serum antibody binding reactivity. However, the patterns of antibody binding can be complex, as patient allosera may contain multiple HLA-specific antibodies due to various sensitizing events such as pregnancy, transfusions, or previous transplant HLA mismatches. Other approaches, use single antigen cell lines to absorb and elute antibodies. However, generating these cell lines is costly, time-consuming, and will present variable levels of HLA expression. Another approach introduced an adsorption/elution protocol using crossmatch cells to enhance HLA-specific antibody detection. However, this protocol relies on the availability of donor cells with the specific HLA variant of interest, posing availability challenges, especially for antibody definition in highly sensitized individuals. Additionally, the presence of numerous HLA and non-HLA molecules on cell surfaces complicates the subsequent identification and analysis of isolated HLA-specific antibodies.

Existing methods and systems for identifying and verifying eplets suffer from a number of shortcomings and there is an ongoing need and desire for improved methods and systems for facilitating analysis of HLA epitopes.

### SUMMARY

In one aspect, the present disclosure provides a magnetic bead conjugated to an HLA or portion thereof, e.g., extracellular domain.

In another aspect, the present disclosure provides a composition that includes a plurality of magnetic beads, wherein each magnetic bead is conjugated to an HLA or portion thereof, e.g., extracellular domain, and wherein the HLA includes at least 20, 30, 40, 50 or more of those set forth in Tables 1-6.

In certain aspects, the disclosure provides a method of isolating HLA-specific antibodies using the composition of the disclosure. In embodiments, the method includes:
contacting the composition of the disclosure with a liquid sample to specifically bind antibodies present in the liquid sample to HLA immobilized on magnetic beads;
separating magnetic beads having antibodies bound to HLA of the magnetic beads from the liquid sample; and
isolating the antibodies bound to magnetic beads.

In related aspects, the disclosure provides a method of isolating DSA from a serum sample. In embodiments, the method includes:
contacting the composition of the disclosure with a serum sample of a putative transplant recipient to specifically bind DSA present in the serum sample to HLA immobilized on magnetic beads;
separating magnetic beads having DSA bound to HLA of the magnetic beads from the serum sample; and
isolating the DSA bound to magnetic beads.

In another aspect, the present disclosure provides a method of assessing immunologic compatibility between a potential transplant donor and a transplant recipient. In embodiments, the method includes:
isolating donor specific antibodies (DSA) from a serum sample from a potential transplant recipient according to the method of the disclosure;
contacting cells of the potential transplant donor with the isolated DSA; and
determining binding of the isolated DSA to the cells of the potential transplant donor, wherein binding of isolated DSA to the cells of the potential transplant donor is indicative of a positive match.

In yet another aspect, the present disclosure includes a method of using magnetic beads coated with HLA antigens to specifically bind to antibodies present in a serum sample from a transplant recipient and to antigens specific to a donor or potential donor.

In still another aspect, the present disclosure provides a method using a magnetic bead conjugated to an HLA to isolate and/or analyze a DSA. In some embodiments, the method includes providing a composition or a magnetic bead of the disclosure; binding an HLA of the composition or magnetic bead to DSA in a liquid sample; and isolating the DSA bound to the HLA from the liquid sample. In various embodiments, the method further includes performing epitope analysis to identify HLA eplets, and/or performing flow analysis on cells with different HLA antigen expression and presentation, and characterizing serological reactions of different HLA alleles, and/or performing a binding kinetics assay to obtain association, dissociation, and binding affinity parameters.

### BRIEF DESCRIPTION OF THE DRAWINGS

For a fuller understanding of the nature and advantages of the present invention, reference should be made to the following detailed description taken in connection with the accompanying drawings. It is appreciated that these drawings depict only typical embodiments of the invention and are therefore not to be considered limiting of its scope. The disclosure will be described and explained with additional specificity and detail through the use of the accompanying drawings.

FIGS. 1A-1B presents data depicting HLA Coated Magnetic Bead Elution MFI to Pre-treatment MFI ratios distribution. 1,329 adsorption elution assays were performed on sera with MFI ranged from 55 to 29,271 (Table 7). The results were grouped into five bins based on pre-treatment MFI of the sera, ranging from 0-600, 600-2500, 2500-5000, 5000-10000, and 10000-30000. FIG. 1A is a violin plot of distribution of ratios for each bin. Number of adsorption and elution assays performed for each pre-treatment sera range is indicated on top as N. Horizontal dashed line indicates the point where Elution MFI equals Pre-treatment MFI. FIG. 1B is a table showing ratio values for first quartile, second (median) quartile, and third quartile for each bin.

FIG. 2 is a graph illustrating HLA coated magnetic bead isolated HLA-specific antibodies from sera. Serum S10160C was adsorbed and eluted with MagSort™ A0101, MagSort™ A0201, MagSort™ B1301, or MagSort™ C0202 targeting HLA-A*01:01, HLA-A*02:01, HLA-B*13:01, or HLA-C*02:02 respectively. LABScreen™ MFI heatmap for pre-treatment serum S10160C (S10160C Pre), isolated HLA-A*01:01-specific antibodies (A*01:01 El), isolated HLA-A*02:01-specific antibodies (A*02:01 El), isolated HLA-B*13:01-specific antibodies (B*13:01 El), and isolated HLA-C*02:02-specific antibodies (C*02:02 El) was visualized. Heatmap MFI scale is from 0-8000.

FIGS. 3A-3B is a series of graphs illustrating HLA coated magnetic bead isolated and depleted HLA-specific antibodies sera. FIG. 3A depicts Serum F1 that were adsorbed and eluted with MagSort™ C1502 targeting HLA-C*15:02. LABScreen™ MFI heatmap for pre-treatment serum F1 (F1 Pre), post-treatment serum F1 (C*15:02 Post), and isolated HLA-C*15:02-specific antibodies (C1502 El) was visualized. FIG. 3B depicts Serum S10544 that were adsorbed and eluted with MagSort™ DQ0401A0201 targeting HLA-DQA1*02:01/DQB1*04:01. LABScreen™ MFI heatmap for pre-treatment serum S10544, post-treatment serum S10544 (DQA1*02:01/DQB1*04:01 Post), and isolated HLA-DQA1*02:01/DQB1*04:01-specific antibodies (DQA1*02:01/DQB1*04:01 El) was visualized. Heatmap MFI scale is from 0-8000.

FIGS. 4A-4D is a series of graphs evidencing that sera dilution allows depletion of HLA-specific antibodies in post-treatment sera. Serum S11181 and serum F2 and their dilution (neat, 10, 50, and 100-fold) were adsorbed and eluted with HLA-B*73:01 and HLA-DQA1*01:03/DQB1*06:03 specific magnetic beads respectively. FIG. 4A is a graph depicting Serum S11181's HLA-B*73:01 MFI signals at different dilutions. Top line is pre-treatment serum S11181. Bottom line is post-treatment serum S11181. Middle line is isolated HLA-B*73:01-specific antibodies. FIG. 4B is a graph depicting Serum F2's HLA-DQA1*01:03/DQB1*06:03 MFI signals at different dilutions. Top line within the boxed area is pre-treatment serum F2. Bottom line within the boxed area is post-treatment serum F2. Middle line within the boxed area is isolated HLA-DQA1*01:03/DQB1*06:03-specific antibodies. FIG. 4C is a LABScreen™ MFI heatmap for pre-treatment serum S11181, post-treatment serum S11181, and isolated HLA-B*73:01-specific antibodies and the corresponding 10-fold dilution was visualized. FIG. 4D is a LABScreen™ MFI heatmap for pre-treatment serum F2, post-treatment serum F2, and isolated HLA-DQA1*01:03/DQB1*06:03-specific antibodies and the corresponding 10-fold dilution was visualized. Heatmap MFI scale is from 0-8000.

FIGS. 5A-5C is a series of representations evidencing that isolated HLA-C*16:01-specific antibodies from Serum-14 positively stained HLA-C*16:01 and HLA-C*16:02 donor cells. FIG. 5A is a LABScreen™ MFI heatmap for pre-treatment serum 14 (14 Pre), post-treatment serum 14 (C*16:01 Post), and isolated HLA-C*16:01-specific antibodies (C*16:01 El). The HLA-C*16:01 MFI signal of the isolated antibodies is ˜ 10,000. Heatmap MFI scale is from 0-10000. FIG. 5B is a table depicting HLA typing for Donor D1 (HLA-C*16:01), D2 (HLA-C*16:01), and D3 (Negative) cells. FIG. 5C are graphs depicting flow cytometry crossmatching (FCXM) on D1, D2, and D3 cells. AMESF of T-cell FCXMs were 2,977 (weak positive) and 3,219 (weak positive) for D1 and D2 cells respectively. FCXM cut off was defined as: 4000>ΔMESF21000 as weak positive and ΔMESF ≥4,000 as positive.

FIGS. 6A-6C is a series of representations evidencing that isolated HLA-C*12:03-specific antibodies from Serum-13 positively stained HLA-C*16:01 and HLA-C*16:02 donor cells. FIG. 6A is a LABScreen™ MFI heatmap for pre-treatment serum 13 (13 Pre), post-treatment serum 13 (C1203 Post), and isolated HLA-C*16:01-specific antibodies (C1203 El). The HLA-C*16:01 and HLA-C*16:02 MFI signal of the isolated antibodies is ˜ 10,000 and ˜20,000 respectively. Heatmap MFI scale is from 0-20000. FIG. 6B is a table depicting HLA typing for Donor D1 (HLA-C*16:01), D2 (HLA-C*16:02), and D3 (Negative) cells. FIG. 6C are graphs depicting flow cytometry crossmatching (FCXM) on D1, D2, and D3 cells. ΔMESF of T-cell FCXMs were 9,735 and 25,819 for D1 (HLA-C*16:01) and D2 (HLA-C*16:02) cells respectively. ΔMESF ≥4,000 was defined as positive.

FIGS. 7A-7E is a series of representations depicting comparison between pre-treatment MFI and elution MFI for sera with different pre-treatment MFI range. FIG. 7A is a violin plot depicting sera with pre-treatment MFI between 0-600. FIG. 7B is a violin plot depicting sera with pre-treatment MFI between 600-2500. FIG. 7C is a violin plot depicting sera with pre-treatment MFI between 2500-5000. FIG. 7D is a violin plot depicting sera with pre-treatment MFI between 5000-10000. FIG. 7E is a violin plot depicting sera with pre-treatment MFI between 10000-30000. Number of adsorption and elution performed for each pre-treatment sera range are indicated at the bottom as N. FIG. 7F is a table showing elution MFI values for first quartile, second (median) quartile, and third quartile for each bin.

FIG. 8 is a heatmap depicting HLA coated magnetic bead isolated HLA-specific antibodies from sera with pre-treatment MFI between 600-2500. Serum S10270C was adsorbed and eluted with MagSort™ A0101 targeting HLA-A*01:01. LABScreen™ MFI heatmap for pre-treatment serum S10270C (S10270C Pre) and isolated HLA-A*01:01-specific antibodies (A*01:01 El) was visualized. Heatmap MFI scale is from 0-500.

FIGS. 9A-9F is a series of graphs depicting representative correlations between MFI signals from the cell method and MFI signals from the magnetic bead method of the disclosure. FIG. 9A depicts correlations for isolated HLA-A*01:01-specific antibodies from Serum S10607. FIG. 9B depicts correlations for isolated HLA-B*08:01-specific antibodies from Serum S10570E. FIG. 9C depicts correlations for isolated HLA-DPA1*01:03/DPB1*02:01-specfic antibodies from Serum S10758.

FIG. 9D depicts correlations for isolated HLA-DPA1*01:03/DPB1*02:01-specific antibodies from Serum S10847. FIG. 9E depicts correlations for isolated HLA-DQA1*01:01/DQB1*05:01-specfic antibodies from Serum S10629C. FIG. 9F depicts correlations for isolated HLA-DQA1*01:01/DQB1*05:01-specfic antibodies from Serum S10261B. Pearson correlation, Spearman correlation, coefficient of determination (R2), and calculated p-values are displayed in each scatter plot. Linear regressions on correlation data are visualized as black lines.

FIGS. 10A-10C is a series of heatmaps depicting isolated HLA-specific antibodies MFI signals comparison between the cell method and the magnetic bead method of the disclosure. FIG. 10A is a LABScreen™ MFI heatmap for pre-treatment serum S10778A, isolated HLA-B*08:01-specific antibodies using the cell method (RC B*08:01 El), and isolated antibodies using the magnetic bead method of the disclosure (MagSort™ B*08:01 El) were visualized. FIG. 10B is a LABScreen™ MFI heatmap for pre-treatment serum S10507E, isolated HLA-A*01:01-specific antibodies using the cell method (RC A*01:01 El), and isolated antibodies using the magnetic bead method of the disclosure (MagSort™ A*01:01 El) were visualized. FIG. 10C is a LABScreen™ MFI heatmap for pre-treatment serum S10629C, isolated HLA-DQA1*01:01/DQB1*05:01-specific antibodies using the cell method (RC DQA1*01:01/DQB1*05:01 El), isolated HLA-DQA1*01:01/DQB1*05:01-antibodies using the magnetic bead method of the disclosure (MagSort™ DQA1*01:01/DQB1*05:01 El), and isolated HLA-DQA1*02:01/DQB1*02:01-specific antibodies using the magnetic bead method of the disclosure (MagSort™ DQA1*02:01/DQB1*02:01 El) were visualized. Heatmap MFI scale is from 0-8000.

FIGS. 11A-11B is a series of representations evidencing eplet discovery on isolated HLA-A*01:01-specific antibodies from Serum-S10160C. FIG. 11A is a LABScreen™ MFI heatmap for pre-treatment serum S10160C (S10160C Pre), isolated HLA-A*01:01-specific antibodies using the cell method (RC A*01:01 El), and isolated HLA-A*01:01-specific antibodies using the magnetic bead method of the disclosure (MagSort™ A*01:01 El). FIG. 11B (left panel) shows molecular visualization for 163R/L167G/S eplet on HLA-A*01:01 (RCSB: 3BO8). Peptide is in stick model, and eplet is in sphere model. FIG. 11B (right panel) is a multiple sequence alignment of HLA antigens using Clustal Omega™. Antigens with positive signals are highlighted with an arrow. The alignment suggests that the corresponding eplet is 163R/L 167G/S.

### DESCRIPTION

Before describing various embodiments of the present disclosure in detail, it is to be understood that this disclosure is not limited to the parameters of the particularly exemplified systems, methods, apparatus, products, processes, consumables, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

The term “comprising” which is synonymous with “including,” “containing,” “having” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “method” includes one, two, or more methods.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although a number of methods and materials similar or equivalent to those described herein can be used in the practice of the present disclosure, the preferred materials and methods are described herein.

To address the challenges mentioned above, the inventors developed magnetic beads coated with single HLA variants (One Lambda™ MagSort™). In embodiments, the HLA coated magnetic beads are used to treat sera with specific HLA variants (each bead coated with a single HLA) to generate pre-treatment sera, as well as post-treatment sera and isolated HLA-specific antibodies. The utility of these beads is described herein for at least the following applications and/or methods, alone or in any combination: isolation of HLA-specific DSAs from complex sera, enabling the unique identification of these DSAs based on their HLA binding pattern, which is especially valuable for determining the eplet-specificity of these DSAs; evaluating the quantity and/or binding strength of HLA-specific antibodies by examining the signal reduction in post-treatment sera alongside the corresponding signal in the isolated DSA fractions at varying dilutions; and performing FCXM to assess HLA-specific antibody reactivity against specific donor cells using HLA coated magnetic bead sorted sera.

### HLA Antigens

Classical HLA are categorized based on sequence and structural homology into 11 loci, namely those containing the Class I HLA alleles: HLA-A, HLA-B and HLA-C, and those loci containing the Class II HLA alleles: HLA-DRB1, HLA-DRB3, HLA-DRB4, HLA-DRB5, HLA-DQA1, HLA-DQB1, HLA-DPA1 and HLA-DPB1. Within each locus, there are conserved and variant positions.

In various embodiments, the disclosure contemplates use of any HLA for immobilization on a magnetic bead. Examples of known HLA that may be immobilized on magnetic beads are available from the IMGT/HLA database (on the World Wide Web at ebi.ac.uk/ipd/imgt/hla/alleles/). It will be appreciated that variants and truncations of these exemplary HLA may also be immobilized on magnetic beads. Additionally, specific examples of HLA utilized in the examples of the disclosure are set forth in Tables 1-6.

TABLE 1
HLA-A

| Antigen | Ser |
|---|---|
| A0101 | A1 |
| A0201 | A2 |
| A0301 | A3 |
| A1101 | A11 |
| A2402 | A24 |
| A2501 | A25 |
| A2901 | A29 |
| A3001 | A30 |
| A3303 | A33 |
| A6801 | A68 |

TABLE 2
HLA-B

| Antigen | Ser |
|---|---|
| B0702 | B7 BW6 |
| B0801 | B8 BW6 |
| B1301 | B13 BW4 |
| B1501 | B62 BW6 |
| B4402 | B44 BW4 |
| B4601 | B46 BW6 |
| B5301 | B53 BW4 |
| B5701 | B57 BW4 |
| B6701 | B67 BW6 |
| B7301 | B73 BW6 |

TABLE 3
HLA-C

| Antigen | Ser |
|---|---|
| C0102 | CW1 |
| C0202 | CW2 |
| C0303 | CW9 |
| C0304 | CW10 |
| C0401 | CW4 |
| C0501 | CW5 |
| C0702 | CW7 |
| C1203 | CW12 |
| C1502 | CW15 |
| C1601 | CW16 |
| C1701 | CW17 |

TABLE 4
HLA-DR

| Antigen | Ser |
|---|---|
| DRB1*0101 | DR1 |
| DRB1*0301 | DR17 |
| DRB1*0401 | DR4 |
| DRB1*0404 | DR4 |
| DRB1*0701 | DR7 |
| DRB1*0801 | DR8 |
| DRB1*1101 | DR11 |
| DRB1*1201 | DR12 |
| DRB1*1301 | DR13 |
| DRB1*1501 | DR15 |
| DRB1*1601 | DR16 |

TABLE 5
HLA-DQ

| Antigen | Ser |
|---|---|
| DQB1*0201 DQA1*0201 | DQ2 |
| DQB1*0202 DQA1*0201 | DQ2 |
| DQB1*0301 DQA1*0201 | DQ7 |
| DQB1*0302 DQA1*0201 | DQ8 |
| DQB1*0303 DQA1*0201 | DQ9 |
| DQB1*0401 DQA1*0201 | DQ4 |
| DQB1*0501 DQA1*0101 | DQ5 |
| DQB1*0602 DQA1*0101 | DQ6 |
| DQB1*0603 DQA1*0103 | DQ6 |

TABLE 6
HLA-DP

| Antigen | Ser |
|---|---|
| DPB1*0101 DPA1*0103 | DP1 |
| DPB1*0201 DPA1*0103 | DP2 |
| DPB1*0301 DPA1*0103 | DP3 |
| DPB1*0401 DPA1*0103 | DP4 |
| DPB1*0402 DPA1*0103 | DP4 |
| DPB1*1101 DPA1*0103 | DP11 |
| DPB1*1701 DPA1*0201 | DP17 |
| DPB1*1901 DPA1*0103 | DP19 |

### HLA Coated Magnetic Beads

As used herein, the term “magnetic bead” refers to a bead support including material capable of being attracted to a magnetic field. In many instances, magnetic beads will comprise one or more magnetic metals (e.g., iron, cobalt, nickel, and the like). Examples of commercially available magnetic beads are Dynabeads™, such as Dynabeads™ M-280 tosylactivated magnetic beads (Thermo Fisher Scientific).

Specific HLA antigens can be linked to the magnetic beads by any technique known to those skilled in the art. Further, HLAs can be directly or indirectly linked to the magnetic beads. In some embodiments, HLAs are directly linked to the magnetic beads by passive absorption, chemical coupling or by chemical linkage through a tail element added to the HLA.

In some embodiments, HLA coated magnetic beads are mixed into a buffer solution, for example, phosphate buffered saline (PBS) containing approximately 1% bovine serum albumin, ovalbumin, casein, or other blocking protein at a concentration of approximately 20 mg of magnetic beads per ml of buffer solution.

In embodiments, the composition of the present disclosure includes magnetic beads coated with a single type of HLA or may include a panel of magnetic beads including a plurality of magnetic beads, wherein the plurality includes single antigen coated magnetic beads of different HLA. In some embodiments, a single magnetic bead may be coated with more than 1, 2, 3, 4 or 5 different HLA. As used herein, a “panel” means a collection of one or more magnetic beads, each coated with a different HLA, or compositions of the disclosure (e.g., a magnetic bead having an immobilized HLA) which may be divided into specific categories or sub-panels (e.g., specific HLA classes, sub-classes (e.g., HLA-A, HLA-B, HLA-C, HLA-DQ and the like), or otherwise) and which may be assayed simultaneously or separately, optionally at different times.

### Isolation of Antibodies the Bind Specific HLA

In certain aspects, the disclosure provides a method of isolating HLA-specific antibodies. In embodiments, the method includes:
contacting the composition of the disclosure with a liquid sample to specifically bind antibodies present in the liquid sample to HLA immobilized on magnetic beads;
separating magnetic beads having antibodies bound to HLA of the magnetic beads from the liquid sample; and
isolating the antibodies bound to magnetic beads.

In related aspects, the disclosure provides a method of isolating DSA from a serum sample. In embodiments, the method includes:
contacting the composition of the disclosure with a serum sample of a putative transplant recipient to specifically bind DSA present in the serum sample to HLA immobilized on magnetic beads;
separating magnetic beads having DSA bound to HLA of the magnetic beads from the serum sample; and
isolating the DSA bound to magnetic beads.

In various embodiments, the antibodies bound to magnetic beads are eluted and the magnetic beads are removed from the sample to generate an isolated population of unbound antibodies, which may optionally be separated by type, analyzed and/or used in further applications as described herein. For example, the antibodies may be used to perform analyses such as epitope analysis, crossmatching analysis, and/or antibody binding kinetic analysis.

In some embodiments, a serum sample from an individual who has received or will receive an organ transplant is mixed at a concentration of about 1:1 to 1:1,000 with HLA coated magnetic beads in buffer solution as described above in a test tube and incubated at about 4° to 30° C. for at least 30 minutes to permit binding of antibodies in the serum with specific HLA molecules.

After incubation, a magnet is applied to the outside of the tube and the fluid contents including any unbound antibodies are removed while the magnet remains in place. Upon releasing the magnet, the remaining magnetic beads are washed at least twice with buffer solution, each time using magnetic force applied to the outside of the tube to retain the magnetic beads. Alternatively, the magnetic beads may be washed once for at least 30 minutes at about 4° to 30° C.

Antibodies bound to HLA coated magnetic beads may be eluted by any known method. For example, the magnetic beads may be exposed to a solution of 0.1 M glycine-HCl at pH 2.5-3.0 containing 75 mM NaCl for about 20-30 minutes at room temperature, or they may be treated with a denaturing buffer comprising guanidine-HCl at a concentration of 2-6 M, urea at a concentration of 2-8 M, and 0.5%-2% SDS. Alternatively, an ionic buffer comprising LiCl (5 M), MgCl2 (3.5 M), KCl (3.0-3.5 M), NaI (2.5 M), KI (2.5 M), sodium thiocyanate (0.2-3.0 M), and/or NaCl (2-5 M) at pH 7-8 may be used. Other types of buffers are also effective including sodium citrate/glycine-HCl at pH of 2-4, or a mixture of triethylamine/NaOH/glycine-NaOH at a pH of 10-12.

Eluted antibodies may be neutralized, for example with 1M Tris-HCl at pH 9, or dialyzed in PBS at pH 7-8, preferably with 1% bovine serum albumin, ovalbumin, casein, or other blocking protein.

### Isolated HLA-Specific Antibody Identification and Epitope Analysis

In embodiments, the methodology of the disclosure further includes identification and use of HLA-specific antibodies isolated by the methods disclosed herein to perform epitope analysis, including identification of epitopes and/or eplets.

General knowledge of the interface between an Ab's binding region (paratope) and its cognate Ag's binding region (epitope) is based on structural information. Although the interface covers a wide area of surface residues, for example up to 25 amino acids in a 15 Å radius, the so-called structural epitope, it is generally accepted that contact point(s) of an Ag interacting with the complementarity-determining region 3 (CDR3) of the Ab heavy chain (HC) determines specificity, while additional contact points between Ag and Ab contribute to affinity. Such a specificity-defining epitope is termed functional epitope which occupies an area of less than 3.5 Å radius. In the case of HLA molecules, due to the high overall sequence and structural homology, a single amino acid polymorphism could define a functional epitope. A functional epitope determines the specificity of interaction with a paratope, and therefore the immunogenicity of a mismatch. Accordingly, Ab-verified functional epitopes are expected to be associated with higher risk of immunogenicity, although the verification methodology has not been standardized.

A functional epitope of HLA, by definition, is specific to a particular paratope of a monoclonal antibody (mAb). Such a functional epitope can be carried by a single allele or by multiple alleles. Because of their polymorphic nature, different HLA molecules carrying the same functional epitope could bind to the same mAb at a very different affinity depending on the overall composition of a complete structural epitope. On the other hand, a single mAb could recognize a single functional epitope on one allele and potentially a different functional epitope at a similar location on another allele. A functional epitope is necessary but insufficient by itself to support a measurable interaction between a specific Ag-Ab pair. A functional epitope combined with the structural epitope which covers the remainder of the interface with the mAb, defines a complete epitope which provides both specificity and affinity of a binding event.

Antibodies bound to magnetic beads coated with a specific HLA molecule represent mono-specific antibodies separated from other antibodies which react to different antigens in a polyclonal serum sample. In embodiments, the eluted mono-specific antibodies may be used for analysis of epitopes recognized by the antibodies using an antigen microparticle screening assay.

As used herein, an ‘antigen microparticle screening assay’ means a microparticle based assay utilizing known HLA (e.g., publicly available Class I or Class II, common and well-documented (CWD) HLA) that are immobilized on beads and which have a detectable label such that HLA-specific antibodies can be identified.

Various detectable labels which are known in the art may be utilized in combination with various types of beads, e.g., microparticles. As used herein, a “microparticles” refers to microparticles, microbeads, beads or microsphere of any material, e.g., silica, gold, latex, polymers such as polystyrene, polysulfone and polyethyl, or hydrogel. Additional exemplary microparticles are encoded with the dyes and the oligonucleotides are immobilized to the encoded microparticles, The microparticles used in the methods of the invention are commercially available from sources such as Luminex Inc., Invitrogen (Carlsbad, Calif.), Polysciences Inc. (Warrington, Pa.) and Bangs Laboratories (Fishers, Ind.) to name a few.

In various embodiments, the microparticles include a detectable label or another identifying characteristic. The microparticles may comprise a single fluorescent dye or multiple fluorescent dyes. In one embodiment, the microparticles are internally labeled with fluorescent dyes and contain surface carboxyl groups for covalent attachment of biomolecules. In another embodiment, the microparticles are internally labeled with fluorescent dyes and contain a surface layer of Avidin for near covalent binding of biotin and biotinylated ligands. In another embodiment, the microparticles may comprise a combination of different dyes, such as a fluorescent and a nonfluorescent dye. For example, the microparticles may be labeled with E)-5-[2-(methoxycarbonyl)ethenyl]cytidine, which is a nonfluorescent molecule, that when subjected to ultraviolet (UV) irradiation yields a single product, 3-β-D-ribofuranosyl-2,7-dioxopyrido[2,3-d]pyrimidine, which displays a strong fluorescence signal. In another embodiment, the microparticles may comprise bar codes as an identifiable characteristic as described in U.S. Patent Publication No. US 20070037195.

In another embodiment, the microparticles may be nanocrystals or quantum dots. These nanocrystals are substances that absorb photons of light, then re-emit photons at a different wavelength (fluorophores). In addition, additional florescent labels, or secondary antibodies may be conjugated to the nanocrystals. These nanocrystals are commercially available form sources such as Invitrogen and Evident Technologies (Troy, N.Y.).

In some embodiments, the antigen microparticle screening assay is a LABScreen™ single antigen bead assay (One Lambda, Thermo Fisher Scientific). The LABScreen™ single antigen bead ExPlex™ assays use a panel of color-coded microbeads, each coated with a purified HLA antigen, specifically 151 Class I or 119 Class II common and well-documented (CWD) HLA antigens. In accordance with product instructions for use, serum containing the eluted HLA-specific antibody, e.g., DSA, is incubated with LABScreen™ beads to allow binding to any antigens containing recognized epitopes. Following incubation, the beads are washed and bound antibodies are labeled with phycoerythrin (PE)-conjugated goat anti-human IgG. Fluorescent emission from the PE labeled anti-human IgG antibodies bound to each bead is detected using LABScan™ 100, LABScan™ 200 or LABScan3D™ systems (One Lambda, Thermo Fisher Scientific).

In embodiments, epitope analysis includes identification of eplets. The current prevailing definition of a functional epitope refers to the HLA eplet system defined by HLAMatchmaker™ where each potential functional epitope is theoretically predefined as an eplet that comprises the minimal amino acid configuration within 3-3.5 Å needed to induce an Ab response. Some eplets are “Ab-verified” while others are not (see, World Wide Web at epregistry.com.br/). However, deficiencies have been described in adopting the eplet system, which is why instead of imputing predefined eplets, this invention considers all individual amino acids at variant positions eps and patterns present in a targeted population(s) regardless of whether they are registered eplets or not.

In embodiments, binding patterns for 151 Class I and 119 Class II alleles are used to identify eplets using HLA Eplet Registry database (available on the World Wide Web at eprigistry.com.br (see, Duquesnoy et al. Int J Immunogenet. 2013; 40(1):54-59. doi: 10.1111/iji.12017)). Based on binding pattern, multiple sequence alignment is performed using, e.g., Clustal Omega™ (see, Madeira et al. Nucleic Acids Res. 2022; 50(W1):W276-W279. doi:10.1093/nar/gkac240) for eplet discovery. In some embodiments, eplets are visualized using, e.g., PyMOL™ (see, Schrodinger. The PyMOL™ Molecular Graphics System, Version 1.8. Published online November 2015.).

### Crossmatching Analysis

In embodiments, the methodology of the disclosure further includes crossmatching analysis to determine compatibility between a donor and donor recipient, e.g., potential donor with a putative transplant recipient. Such analysis may be performed using DSA isolated as described herein, before or after a transplant procedure. As discussed herein, the presence of DSA in a sample from a donor recipient is associated with antibody-mediated rejection (AMR) in transplant patients which adversely impacts long-term survival.

Biological samples that may be utilized in crossmatch testing and analysis include whole blood, blood derivatives, red blood cell concentrates, plasma, serum, fresh frozen plasma, whole blood derived platelet concentrates, apheresis platelets, pooled platelets, intravenous gamma-globulin, cryoprecipitate, cerebrospinal fluid, tissues and cells such as epithelial cells, such as those collected from the buccal cavity, stem cells, leukocytes, neutrophils and granulocytes. The biological samples may be obtained from a human donor of tissue or cells intended for transplantation or a human donor of blood or blood derivatives intended for transfusion. The biological sample may be obtained from a healthy bone marrow donor or a subject of a paternity test. The biological sample may also be obtained from a human subject that is an intended recipient of a transplant or transfusion, or the human subject that is donating the tissue or organ intended for transplantation or transfusion. Alternatively, the biological sample may be obtained directly from tissues or cells that are intended for transplantation in a human recipient. In addition, the biological sample may be obtained from blood or blood derivatives that are intended for transfusion in a human recipient. FCXM using isolated HLA-specific antibodies

In some embodiments, crossmatching analysis includes FCXM using isolated HLA-specific antibodies. In embodiments, an antigen microparticle screening assay is performed to confirm the isolation of HLA-specific antibodies from a sample. Donor cells with target HLA specificity are identified by a genetic typing assay, such as AllType™ FASTplex™ NGS Assay, contacted with isolated HLA-specific antibodies, and antibody binding is analyzed using a flow cytometry technique using fluorescence-based technology.

In some embodiments, 60 μL of serum is diluted 10-fold to 600 μL using Wash Buffer. Diluted sera is treated with 30 μL of magnetic beads (approximately 2.4×10⁶ beads). Isolated antibodies are eluted with 540 μL of Elution Buffer and neutralized with 60 μL of Neutralization Buffer. 10 μL of each post-treatment serum and 10 μL of each eluate are used for LABScreen™ analysis to confirm the isolation of HLA-specific antibodies as described herein.

In some embodiments, donor cells with target HLA specificity are stained with isolated HLA-specific antibodies using standard protocol. Briefly, lymphocytes are isolated at a concentration of 10⁷ cells/mL in PBS+2% FCS. Donor cells were typed using AllType™ FASTplex™ NGS Assay. 88-500 μL of isolated HLA-specific antibodies with known binding toward target HLA were incubated with 15 μL (150,000) isolated cells for 20 minutes at room temperature in the dark on a tabletop rotator with gentle rotation. Cells were centrifuged at 2000 g for 1.5 minutes, flicked gently, and blotted on bench pad or paper towels. Next, cells are washed 3 times with 200 μL PBS+2% FCS by centrifugation at 2,000 g for 1.5 minutes. 50 μL antibody dye cocktail (anti-CD3 PerCP (Thermo Fisher 46-0032-82), anti-CD19 PE (Thermo Fisher 12-0199-42), and anti-IgG FITC (Jackson ImmunoResearch 109-095-003)) is then added and incubated for 5 minutes at room temperature in the dark on a tabletop rotator with gentle rotation. Following incubation, cells were centrifuged at 2,000 g for 1.5 minutes, flicked gently, and blotted on bench pad or paper towels. Cells are washed with 200 μL PBS+2% FCS by centrifugation at 2,000 g for 1.5 minutes. Cells are resuspended in 200 μL PBS+2% FCS and analyzed using an immunocytometry system, such as FacsCanto™ II HTS. Channel shift of FCXM was converted into the shift of Molecules of Equivalent Soluble Fluorophores (ΔMESF) to allow quantitative fluorescence intensity measurements over time and across platforms. FCXM cut off was defined as: 4000>ΔMESF≥1000 as weak positive and ΔMESF ≥4,000 as positive.

In some embodiments, crossmatching analysis includes flow cytometry using isolated HLA-specific antibodies. In embodiments, an antigen microparticle screening assay is performed to confirm the isolation of HLA-specific antibodies from a sample. Cells from a potential donor are contacted with isolated HLA-specific antibodies, with subsequent addition of fluorescently labeled anti-human IgG antibodies, e.g., FITC-labeled goat anti-human IgG antibody. Binding is then analyzed using a flow cytometer to detect the presence/level of fluorescence.

In one embodiment, white blood cells from a potential donor are placed in a buffered saline solution (for example, Dulbecco's phosphate buffered saline containing 2% fetal bovine serum) at a concentration of approximately 100 cells per microliter, and then added to an approximately equal volume of eluted DSA for at least 0.5 hour at about 4° C. Following two washes in buffered saline, the cells are then placed in buffered saline containing labeled anti-human IgG antibodies (for example, FITC-labeled goat anti-human IgG). After at least an hour incubation at about 4° C., a flow cytometer is used to determine the level of DSA binding. A low level of eluted antibody binding is indicative of a low level of DSA in the sample.

### HLA-Specific Antibody Binding Kinetics Analysis

In embodiments, the methodology of the disclosure further includes performing antibody binding kinetics analysis to determine association, dissociation, and binding affinity parameters. It will be appreciated that a number of various techniques for conducting antibody binding kinetics analysis are known in the art and capable of use with the methods of the present disclosure.

In some embodiments, bio-layer interferometry is used to measure kinetics and biomolecular interactions based on wave interference. For example, in one embodiment, Octet™ aminopropylsilane (APS) biosensors (Sartorius) is exposed to about 200 μl of solution containing HLA at approximately 1 μg/1 μl and incubated for about 20 minutes. After washing, the HLA coated biosensor are incubated with eluted DSA for approximately 15 minutes to determine association kinetics using an Octet™ RED96™ instrument (Sartorius). Dissociation kinetics are then determined by incubating the HLA coated biosensor with buffered saline for approximately 15 minutes.

In embodiments, the HLA coated biosensor exposed to DSA is further exposed to labeled anti-human IgG antibody, for example gold-conjugated goat anti-human IgG, to confirm that eluted DSA is of the IgG subtype.

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

### Using Single Antigen Coated Magnetic Beads for Isolation of Specific Antibodies Against HLA

The presence of multiple DSA targeting HLA poses a challenge to transplantation. Various techniques have been developed to isolate DSAs using recombinant cell lines and crossmatch cells. To simplify the extraction of HLA-specific DSAs from complex sera, the inventors introduced magnetic beads with single HLA specificity. Sera were treated with HLA coated magnetic beads, allowing HLA-specific antibodies to bind to the beads, and these specific antibodies were subsequently eluted. Magnetic beads, coated with 59 different HLA variants, underwent testing through 1,329 adsorption/elution processes, demonstrating their effectiveness in adsorbing and eluting HLA-specific antibodies. For sera with pre-treatment trimmed mean fluorescent intensity (MFI) above 2500, 30-200% of the specific MFI signal was recovered in the elution fraction, with a median background MFI signal of 12. The specific binding patterns observed in the eluted fraction can be used for eplet discovery. The binding patterns and strength of isolated antibodies are also comparable between the method utilizing HLA magnetic beads described herein and the cell method. Furthermore, the inventors demonstrated the utility of HLA coated magnetic beads in extracting specific signals from sera for flow cytometry crossmatching, providing a means to assess the reactivity of isolated antibodies against specific donor cells.

### EXPERIMENTAL MATERIALS AND METHODS

### Materials

Single HLA coated magnetic beads (One Lambda™ MagSort™), recombinant cells and immortalized cells expressing specific HLA, LABScreen™ Single Antigen, LABScreen™ Single Antigen ExPlex™, LABScan3D™, and AllType™ FASTplex™ NGS Assay were from One Lambda, Inc. (A Part of Thermo Fisher Scientific Inc.). Sera were sourced from post-transplant patients and HLA sensitized donors, including multiparous females. Sera were prepared according to standard protocol (see, Zhang et al. Hum Immunol. 2017; 78(11-12):699-703. doi: 10.1016/j.humimm.2017.09.001).

### Magnetic Beads

Single HLA coated magnetic beads were generated with either recombinant HLA expressed and purified from cells or native HLA purified from EBV immortalized cell lines as previously described (see, Pei et al. Transplantation. 2003; 75(1):43-49. doi: 10.1097/00007890-200301150-00008). HLA were purified using affinity chromatography with columns attached with anti-HLA Class I or Class II specific monoclonal antibodies. Purified HLA were checked by a panel of monoclonal antibodies and sera specific to particular HLA variants using flow cytometry. It was expected that the density of HLA molecules per bead is higher than the density of HLA molecules per cell based on flow cytometry data.

### Isolation of HLA-Specific Antibodies Using Cells (Cell Method)

Isolations of HLA-specific antibodies were performed as previously described (sec, El-Awar et al. Hum Immunol. 2007; 68(3):170-180. doi: 10.1016/j.humimm.2006.11.006; Liwski et al. Front Genet. 2022; 13:1059650. doi: 10.3389/fgene.2022.1059650; and El-Awar et al. J Immunol Res. 2017; 2017:3406230. doi: 10.1155/2017/3406230). Briefly, 20 μL of each serum was treated with 10×10⁶ cells expressing specific HLA for 45 minutes under gentle rotation at room temperature. The cells were then centrifuged for 5 minutes at 500 g. Each post-treatment serum was transferred to a new tube for analysis. The cells were washed twice for 5 minutes with 1 mL of Dulbecco's phosphate-buffered saline (DPBS) under gentle rotation. Cells were collected by centrifugation at 500 g for 5 minutes. HLA-specific antibodies were eluted by incubating the cells with 20 μL of IgG Elution Buffer (Thermo Fisher 21004) for 15 minutes under gentle rotation at room temperature. The cells were once again centrifuged for 5 minutes at 500 g. Each eluate was transferred to a new tube and neutralized using 2.2 μL of 1 M Tris-HCl pH 9.5. The post-treatment sera and eluted antibodies were used for subsequent analysis.

### Isolation of HLA-Specific Antibodies Using HLA Coated Magnetic Beads

Magnetic beads coated with 59 HLA variants were treated with 11-33 sera per variant (Table 7). The pre-treatment MFI values ranged from 55 to 29271. In some experiments, sera were diluted 10, 50, and 100-fold with 1×PBS. All treatments were done at room temperature.

Table 7: HLA coated magnetic beads were used to adsorb and elute HLA-specific antibodies from 11 to 33 sera per HLA variant. A total of 1,329 adsorption elution assays were performed. Column 1: HLA class I variants used for specific antibody isolation. Column 2: number of sera tested per HLA class I variant. Column 3: HLA class II variants used for specific antibody isolation. Column 4: number of sera tested per HLA class II variant.

| HLA variant | # Sera |
|---|---|
| HLA-A*01:01 | 26 |
| HLA-A*02:01 | 29 |
| HLA-A*03:01 | 26 |
| HLA-A*11:01 | 31 |
| HLA-A*24:02 | 30 |
| HLA-A*25:01 | 33 |
| HLA-A*29:01 | 24 |
| HLA-A*30:01 | 29 |
| HLA-A*33:03 | 24 |
| HLA-A*68:01 | 25 |
| HLA-B*07:02 | 18 |
| HLA-B*08:01 | 20 |
| HLA-B*13:01 | 20 |
| HLA-B*15:01 | 26 |
| HLA-B*44:02 | 21 |
| HLA-B*46:01 | 23 |
| HLA-B*53:01 | 26 |
| HLA-B*57:01 | 28 |
| HLA-B*67:01 | 26 |
| HLA-B*73:01 | 23 |
| HLA-C*01:02 | 14 |
| HLA-C*02:02 | 17 |
| HLA-C*03:03 | 19 |
| HLA-C*03:04 | 15 |
| HLA-C*04:01 | 13 |
| HLA-C*05:01 | 22 |
| HLA-C*07:02 | 13 |
| HLA-C*12:03 | 10 |
| HLA-C*15:02 | 20 |
| HLA-C*16:01 | 11 |
| HLA-C*17:01 | 21 |
| HLA-DPA1*01:03/DPB1*01:01 | 11 |
| HLA-DPA1*01:03/DPB1*02:01 | 14 |
| HLA-DPA1*01:03/DPB1*03:01 | 19 |
| HLA-DPA1*01:03/DPB1*04:01 | 13 |
| HLA-DPA1*01:03/DPB1*04:02 | 14 |
| HLA-DPA1*01:03/DPB1*11:01 | 16 |
| HLA-DPA1*01:03/DPB1*19:01 | 10 |
| HLA-DPA1*02:01/DPB1*17:01 | 10 |
| HLA-DQA1*01:01/DQB1*05:01 | 19 |
| HLA-DQA1*01:01/DQB1*06:02 | 21 |
| HLA-DQA1*01:03/DQB1*06:03 | 20 |
| HLA-DQA1*02:01/DQB1*02:01 | 19 |
| HLA-DQA1*02:01/DQB1*02:02 | 18 |
| HLA-DQA1*02:01/DQB1*03:01 | 33 |
| HLA-DQA1*02:01/DQB1*03:02 | 33 |
| HLA-DQA1*02:01/DQB1*03:03 | 25 |
| HLA-DQA1*02:01/DQB1*04:01 | 17 |
| HLA-DRA1*01:01/DRB1*01:01 | 41 |
| HLA-DRA1*01:01/DRB1*03:01 | 25 |
| HLA-DRA1*01:01/DRB1*04:01 | 32 |
| HLA-DRA1*01:01/DRB1*04:04 | 33 |
| HLA-DRA1*01:01/DRB1*07:01 | 21 |
| HLA-DRA1*01:01/DRB1*08:01 | 30 |
| HLA-DRA1*01:01/DRB1*11:01 | 27 |
| HLA-DRA1*01:01/DRB1*12:01 | 26 |
| HLA-DRA1*01:01/DRB1*13:01 | 33 |
| HLA-DRA1*01:01/DRB1*15:01 | 40 |
| HLA-DRA1*01:01/DRB1*16:01 | 26 |

For downstream LABScreen™ analysis, 20 μL of each serum was treated with 5 μL of HLA coated magnetic beads (approximately 4×10⁵ beads) for 2 hours under gentle rotation. The beads were separated using the DynaMag™-2 Magnet (Thermo Fisher 12321D) for 2 minutes. Each post-treatment serum was transferred to a new tube for downstream analysis. The beads were washed twice with 1 mL of Wash Buffer for 5 minutes each under gentle rotation. Afterward, the beads were separated using a magnet for 2 minutes. HLA-specific antibodies were eluted by incubating the beads with 18 μL of Elution Buffer for 1 hour under gentle agitation. The eluate/beads mixture was separated using a magnet. Each eluate was neutralized using 2 μL of Neutralization Buffer. The post-treatment sera and eluted antibodies were used for subsequent analysis.

Pre-treatment sera, post-treatment sera, and isolated HLA-specific antibodies were analyzed using LABScreen™ Single Antigen and LABScreen™ Single Antigen ExPlex™ panel (LABScreen™ SAB) and LABScan3D™ according to manufacturer instructions (see, Pei et al. Hum Immunol. 1999; 60(12): 1293-1302. doi: 10.1016/s0198-8859(99)00121-4; and Pei et al. Transplantation. 2003; 75(1):43-49. doi: 10.1097/00007890-200301150-00008). MFI values were recorded with the MFI divider setting turned on.

### Eplet Discovery

Binding patterns for 151 Class I and 119 Class II alleles were used to identify eplets using HLA Eplet Registry database (available on the World Wide Web at eprigistry.com.br (sec, Duquesnoy et al. Int J Immunogenet. 2013; 40(1):54-59. doi: 10.1111/iji.12017)). Based on binding pattern, multiple sequence alignment was performed using Clustal Omega™ (see, Madeira et al. Nucleic Acids Res. 2022; 50(W1):W276-W279. doi:10.1093/nar/gkac240) for eplet discovery. Eplets were visualized using PyMOL™ (see, Schrodinger. The PyMOL™ Molecular Graphics System, Version 1.8. Published online November 2015.).

### FCXM Using Isolated HLA-Specific Antibodies

60 μL of each serum was diluted 10-fold to 600 μL using Wash Buffer. Diluted sera were treated as above with 30 μL of magnetic beads (approximately 2.4×10⁶ beads). Isolated antibodies were eluted with 540 μL of Elution Buffer and neutralized with 60 μL of Neutralization Buffer. 10 μL of each post-treatment serum and 10 μL of each eluate were used for LABScreen™ analysis to confirm the isolation of HLA-specific antibodies as described herein.

Donor cells with target HLA specificity were stained with isolated HLA-specific antibodies using standard protocol. Briefly, lymphocytes were isolated at a concentration of 10⁷ cells/mL in PBS+2% FCS. Donor cells were typed using AllType™ FASTplex™ NGS Assay. 88-500 μL of isolated HLA-specific antibodies with known binding toward target HLA were incubated with 15 μL (150,000) isolated cells for 20 minutes at room temperature in the dark on a tabletop rotator with gentle rotation. Cells were centrifuged at 2000 g for 1.5 minutes, flicked gently, and blotted on bench pad or paper towels. Next, cells were washed 3 times with 200 μL PBS+2% FCS by centrifugation at 2,000 g for 1.5 minutes. 50 μL antibody dye cocktail (anti-CD3 PerCP (Thermo Fisher 46-0032-82), anti-CD19 PE (Thermo Fisher 12-0199-42), and anti-IgG FITC (Jackson ImmunoResearch 109-095-003)) was then added and incubated for 5 minutes at room temperature in the dark on a tabletop rotator with gentle rotation. Following incubation, cells were centrifuged at 2,000 g for 1.5 minutes, flicked gently, and blotted on bench pad or paper towels. Washed cells with 200 μL PBS+2% FCS by centrifugation at 2,000 g for 1.5 minutes. Resuspended cells in 200 μL PBS+2% FCS and analyzed using FacsCanto™ II HTS. Channel shift of FCXM was converted into the shift of Molecules of Equivalent Soluble Fluorophores (ΔMESF) to allow quantitative fluorescence intensity measurements over time and across platforms. FCXM cut off was defined as: 4000>ΔMESF≥1000 as weak positive and ΔMESF ≥4,000 as positive.

### Statistical Analysis

Pearson correlation and Spearman rank-order correlation were used to compare trimmed mean fluorescence intensity (MFI) signals of isolated HLA-specific antibodies obtained from cell treatment and HLA coated magnetic bead treatment. To further assess the comparison, linear regression on the correlation data was performed and the coefficient of determinations (R2) were also calculated. p-values for Pearson correlation and Spearman rank-order correlation were calculated using t-test and permutation test respectively.

### Results

### Using HLA Coated Magnetic Beads to Adsorb and Elute HLA-Specific Antibodies

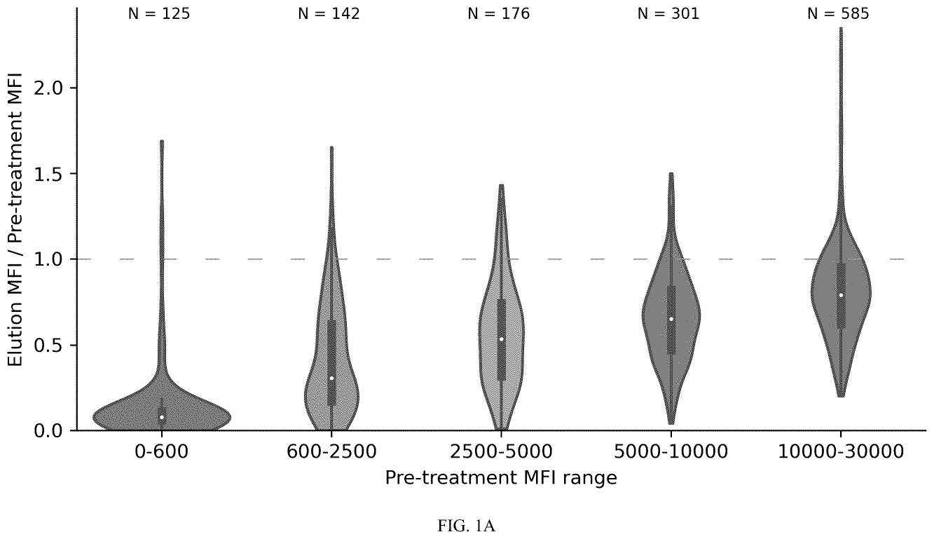

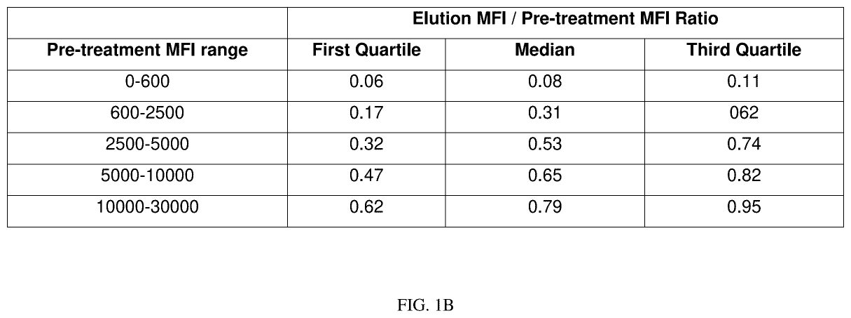

To assess their performance, magnetic beads coated with 59 HLA variants were tested with sera with a wide range of pre-treatment trimmed mean fluorescence intensity (MFI), resulting in a total of 1,329 adsorption/elution processes. The results were grouped into five bins based on pre-treatment MFI of the sera, ranging from 0-600, 600-2500, 2500-5000, 5000-10000, and 10000-30000. Ratios of elution MFI to pre-treatment MFI for each serum were then calculated. The distribution of these ratios for each bin is visualized as a violin plot in FIG. 1A. FIG. 1B displays the ratio values for the first, second (the median), and third quartiles. The median elution MFI to pre-treatment MFI ratios are as follows: 0.08, 0.31, 0.53, 0.65, and 0.79 for the respective MFI bins of 0-600, 600-2500, 2500-5000, 5000-10000, and 10000-30000.

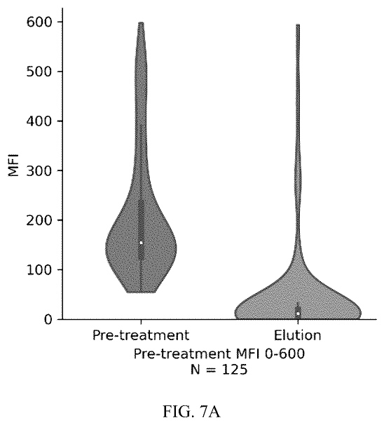

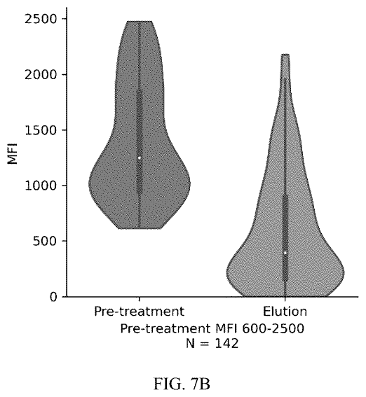

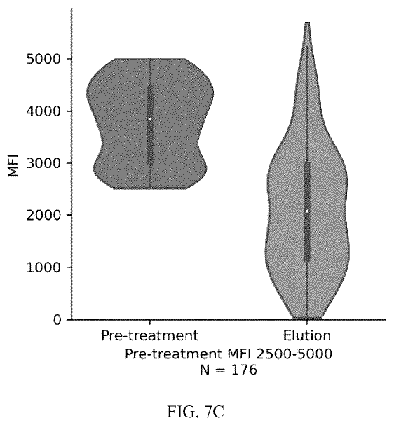

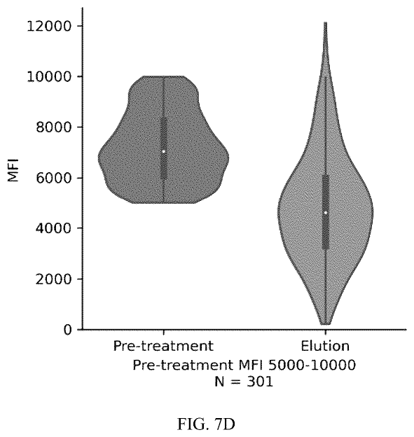

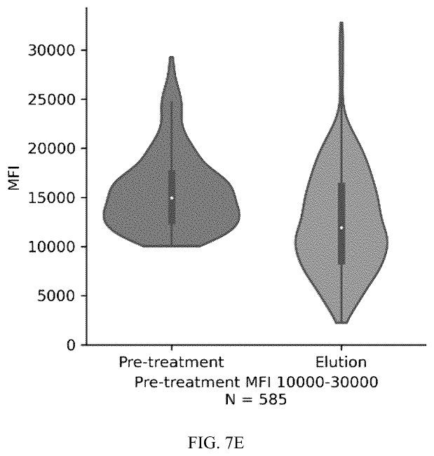

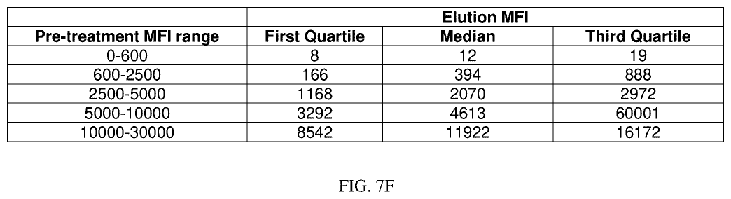

Comparison between pre-treatment sera's MFI and elution MFI is visualized as violin plots in FIG. 7. For sera with pre-treatment MFI between 0-600 and a median MFI of 154, the median elution MFI is 12. For sera with pre-treatment MFI between 600-2500 and a median MFI of 1249, the median elution MFI is 394. For sera with pre-treatment MFI between 2500-5000 and a median MFI of 3845, the median elution MFI is 2070. For sera with pre-treatment MFI between 5000-10000 and a median MFI of 7036, the median elution MFI is 4613. For sera with pre-treatment MFI between 10000-30000 and a median MFI of 14962, the median elution MFI is 11921.

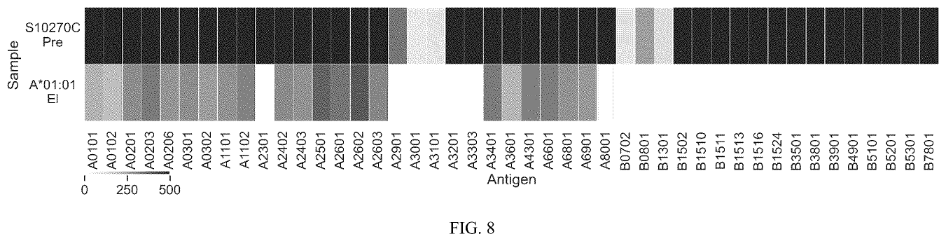

An example for sera with pre-treatment MFI between 600-2500 is shown in FIG. 8. Scrum S10270C was adsorbed and eluted with magnetic beads targeting HLA-A*01:01. Pre-treatment HLA-A*01:01 MFI is 937. Isolated HLA-A*01:01-specific antibodies HLA-A*01:01 MFI is 223. Average HLA-B and HLA-C MFI signal for the isolated antibodies is 8. HLA-A*01:01-specific antibodies binding reactivity corresponds to eplet 151H, 44KM, 149AH, 152HA, 163R, and 163RG.

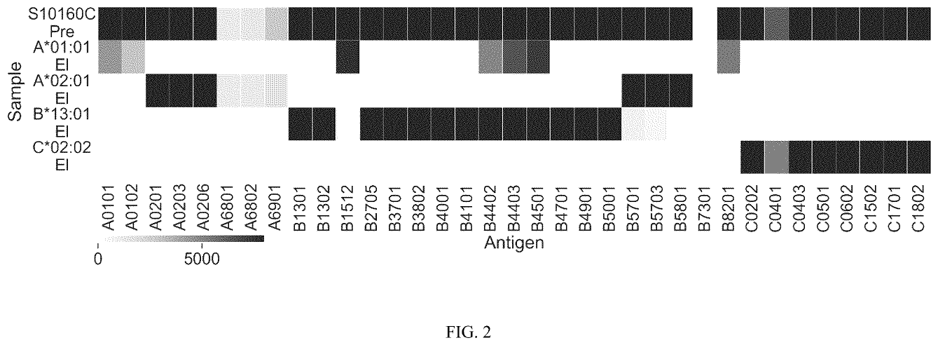

An example where multiple magnetic beads were used to dissect a serum is shown in FIG. 2. Serum S10160C was adsorbed and eluted with magnetic beads targeting HLA-A*01:01, HLA-A*02:01, HLA-B*13:01, and HLA-C*02:02. MFI heatmap for pre-treatment serum S10160C, isolated HLA-A*01:01-specific antibodies, isolated HLA-A*02:01-specific antibodies, isolated HLA-B*13:01-specific antibodies, and isolated HLA-C*02:02-specific antibodies is visualized in FIG. 2. Isolated HLA-A*01:01-specific antibodies binding pattern does not correspond to known eplets. Isolated HLA-A*02:01-specific antibodies binding pattern corresponds to eplet 62GE, 62GK, 107W, and 144TKH. Isolated HLA-B*13:01-specific antibodies binding pattern corresponds to eplet 80TA, 80TLR, 144QL, and 45KE. HLA-C*02:02-specific antibodies binding pattern corresponds to eplet 16S and 80K.

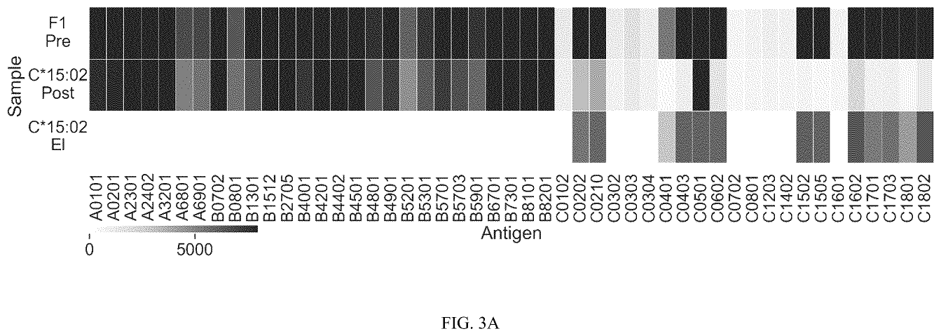

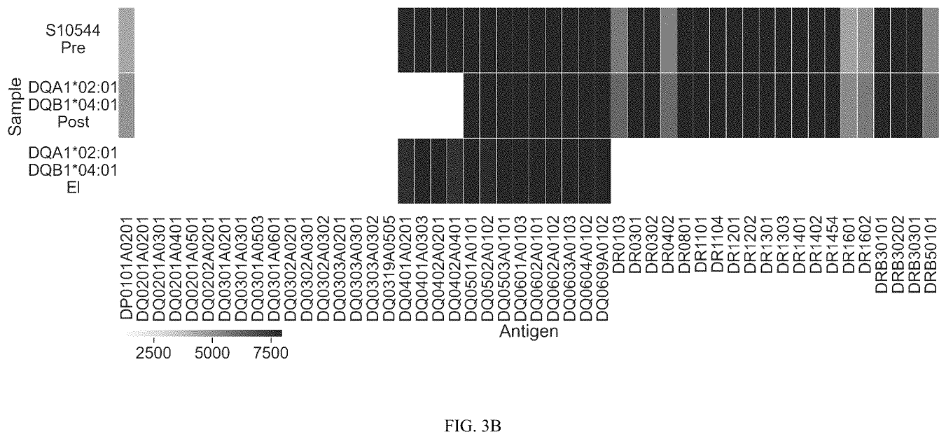

For certain sera, magnetic bead adsorption and elution completely depleted HLA-specific antibodies signals in post-treatment sera, as demonstrated in FIG. 3. Serum F1 was adsorbed and eluted with magnetic beads targeting HLA-C*15:02. MFI heatmap for pre-treatment serum F1, post-treatment serum F1, and isolated HLA-C*15:02-specific antibodies is visualized in FIG. 3A. Post-treatment serum F1 has reduced HLA-C binding reactivity with HLA-C*15:02 and HLA-C*15:05 signal depleted. Isolated HLA-C*15:02-specific antibodies binding reactivity does not include HLA-A or HLA-B signals. HLA-C*15:02-specific antibodies binding reactivity corresponds to eplet 80K.

Serum S10544 was adsorbed and eluted with HLA coated magnetic beads targeting HLA-DQA1*02:01/DQB1*04:01. MFI heatmap for pre-treatment serum S10544, post-treatment serum S10544, and isolated HLA-DQA1*02:01/DQB1*04:01-specific antibodies are visualized in FIG. 3B. Post-treatment serum F1 has depleted HLA-DQA1*02:01/DQB1*04:01, HLA-DQA1*03:03/DQB1*04:01, HLA-DQA1*02:01/DQB1*04:02, and HLA-DQA1*04:01/DQB1*04:02 signals. Isolated HLA-DQA1*02:01/DQB1*04:01-specific antibodies binding reactivity does not include HLA-DP or HLA-DR signals. HLA-DQA1*02:01/DQB1*04:01-specific antibodies binding reactivity corresponds to eplet 55R, 56L, and 74S.

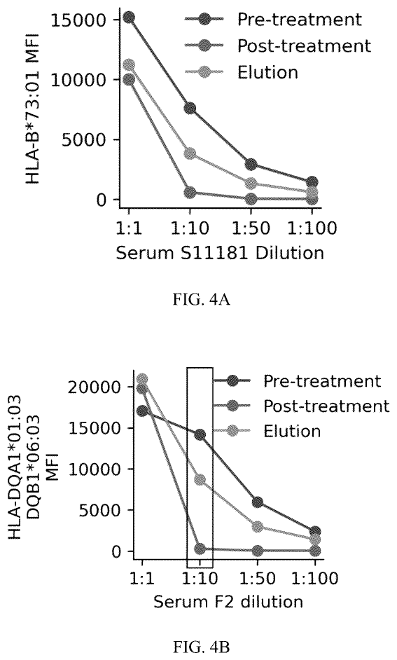

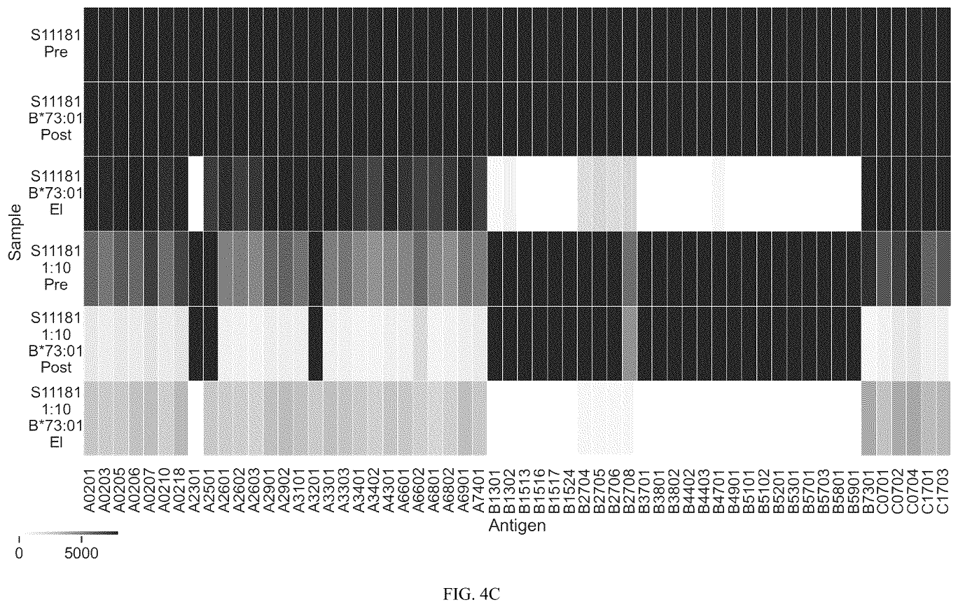

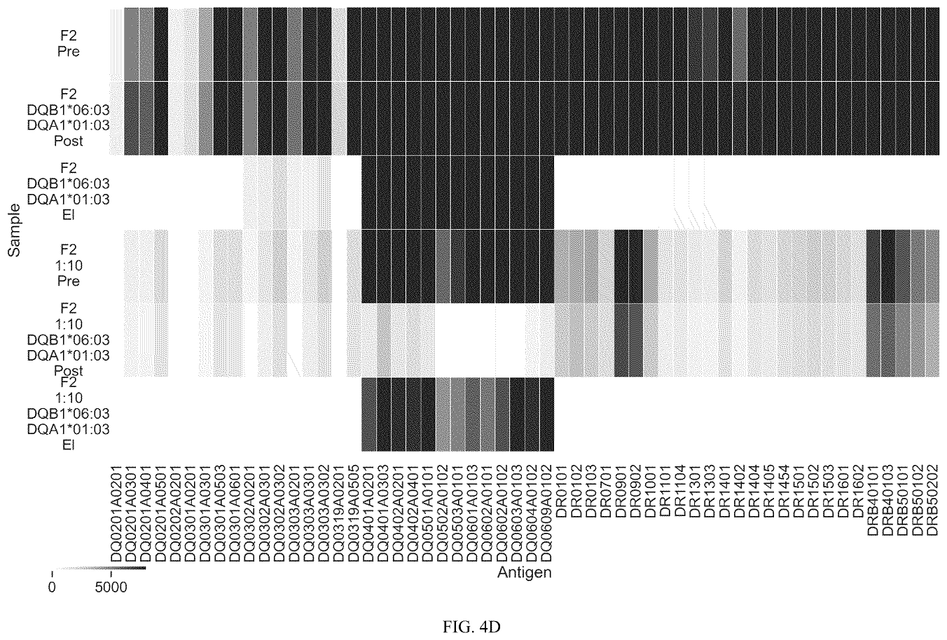

For certain sera, magnetic bead adsorption and elution did not completely deplete HLA-specific antibodies signals in post-treatment sera. For example, serum S11181 and serum F2 were adsorbed and eluted with HLA-B*73:01 and HLA-DQA1*01:03/DQB1*06:03 specific HLA coated magnetic beads respectively. Post-treatment serum S11181 and post-treatment serum F2 still have significant (MFI >10000) HLA-B*73:01 and DQA1*01:03/DQB1*06:03 signals respectively (FIGS. 4A and 4B).

Serum S11181 and serum F2 were diluted 10, 50, and 100-fold. Diluted serum S11181 and serum F2 were adsorbed and eluted with HLA-B*73:01 and HLA-DQA1*01:03/DQB1*06:03 specific HLA coated magnetic beads respectively. HLA-B*73:01 and HLA-DQA1*01:03/DQB1*06:03 signals for pre-treatment, post-treatment, and isolated HLA-specific antibodies from serum S11181 and F2 different dilutions are graphed in FIGS. 4A and 4B. At that 10-fold dilution points, isolated HLA-specific antibodies still give good MFI signals (˜3,000 for HLA-B*73:01 and ˜8,000 for HLA-DQA1*01:03/DQB1*06:03). Above 10-fold dilution points, HLA-B*73:01 and HLA-DQA1*01:03/DQB1*06:03 signals were depleted in their respective post-treatment sera.

MFI heatmap for pre-treatment serum S11181, post-treatment serum S11181, and isolated HLA-B*73:01-specific antibodies and the corresponding 10-fold dilution is visualized in FIG. 4C. MFI heatmap for pre-treatment serum F2, post-treatment serum F2, and isolated HLA-DQA1*01:03/DQB1*06:03-specific antibodies and the corresponding 10-fold dilution is visualized in FIG. 4D.

With serum S11181, both at neat and at 10-fold dilution, the binding reactivity of isolated HLA-B*73:01-specific antibodies corresponds to eplet 253Q and 71KA. In post-treatment serum S11181 at a 10-fold dilution, binding reactivity corresponds to eplet 71TN, 81ALR, and 82LR. Notably, HLA-B*73:01 signal was depleted in post-treatment serum S11181 at a 10-fold dilution. In the case of serum F2 at a 10-fold dilution, binding reactivity of isolated HLA-DQA1*01:03/DQB1*06:03-specific antibodies corresponds to 55R eplet. HLA-DQA1*01:03/DQB1*06:03 signal was effectively depleted in post-treatment serum F2 at a 10-fold dilution, leaving HLA-DR signals behind.

### Comparison Between Cell and HLA Coated Magnetic Bead Adsorption and Elution

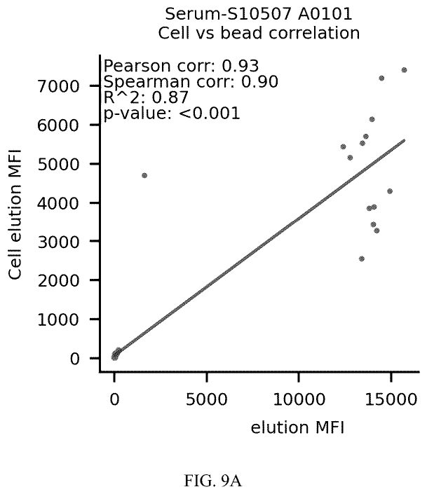

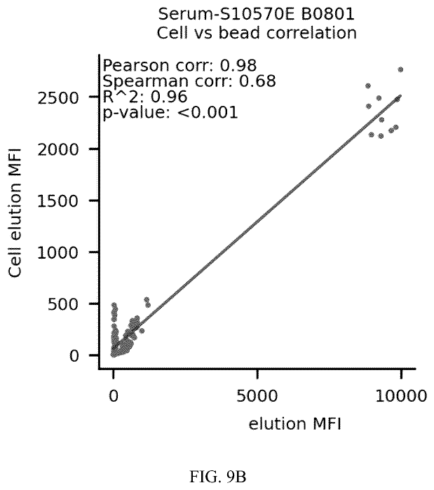

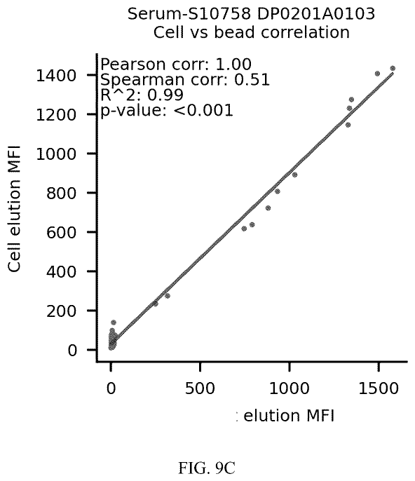

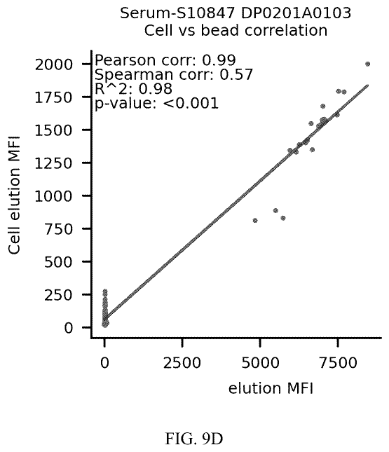

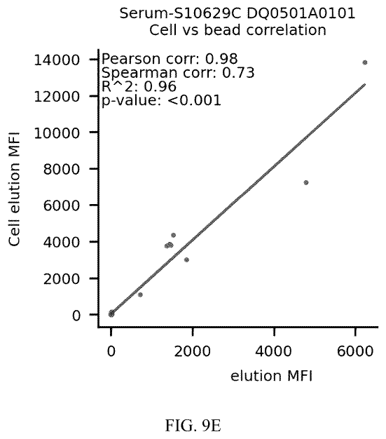

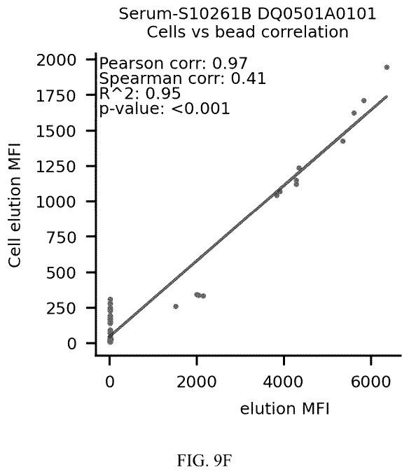

A comparison was made between cell lines and coated magnetic beads regarding their efficacy in isolating HLA-specific antibodies. A total of 27 sera were adsorbed and eluted using either cell lines or magnetic beads expressing or coated with eight HLA antigens: HLA-A*01:01, HLA-B*08:01, HLA-C*12:02, HLA-C*12:03, HLA-C*15:02, HLA-DRA1*01:01/DRB1*11:01, HLA-DPA1*01:03/DPB1*02:01, and HLA-DQA1*01:01/DQB1*05:01. The resulting MFI signals for isolated HLA-specific antibodies on the LABScreen™ panel were obtained. Pearson correlation and Spearman rank-order correlation were calculated between the cell method and the magnetic bead method. Linear regression and coefficient of determination (R2) were used to visualize the correlation strength. Hypothesis testing, with the null hypothesis of no correlation, was conducted using Student's t-test and permutation test, and the results are summarized in Table 8. For the tested sera, Pearson correlation coefficients range from 0.83 to 1.00, Spearman correlation coefficients range from 0.41 to 1.00, and calculated p-values are less than 0.001. Representative correlations between MFI signals from the cell method and the magnetic bead method of the disclosure are visualized in FIG. 9 for HLA-A*01:01, HLA-B*08:01, HLA-DPA1*01:03/DPB1*02:01, and HLA-DQA1*01:01/DQB1*05:01.

Table 8: Correlation of MFI signals for isolated HLA-specific antibodies between the cell method and the magnetic bead method. Column 1: HLA variants used for specific antibody isolation. Column 2: range of Pearson correlation obtained from tested sera. Column 3: range of Spearman correlation obtained from tested sera. Column 4: calculated p-value for Pearson correlation using Student t-test. Column 5: calculated p-value for Spearman correlation using permutation test. Column 6: range of coefficient of determinations (R2) obtained from tested sera. Column 7: number of sera tested.

| | | | | Permutation | | |
|---|---|---|---|---|---|---|
| | Pearson | Spearman | t-test | test | | # Sera |
| HLA variant | Correlation | correlation | p-value | p-value | R² | tested |
| HLA-A*01:01 | 0.93-0.99 | 0.83-0.91 | <0.001 | <0.001 | 0.86-0.87 | 6 |
| HLA-B*08:01 | 0.91-0.98 | 0.64-0.91 | <0.001 | <0.001 | 0.83-0.96 | 5 |
| HLA-C*12:02 | 0.96-1.00 | 0.92-0.98 | <0.001 | <0.001 | 0.95-1.00 | 2 |
| HLA-C*12:03 | 0.96-1.00 | 0.92-0.98 | <0.001 | <0.001 | 0.93-0.99 | 2 |
| HLA-C*15:02 | 0.99-1.00 | 0.82-0.87 | <0.001 | <0.001 | 0.99-0.99 | 2 |
| HLA- | 0.88-0.99 | 0.87-0.98 | <0.001 | <0.001 | 0.77-0.99 | 5 |
| DRA1*01:01/ | | | | | | |
| DRB1*11:01 | | | | | | |
| HLA- | 0.99-1.00 | 0.51-0.57 | <0.001 | <0.001 | 0.98-0.99 | 2 |
| DPA1*01:03/ | | | | | | |
| DPB1*02:01 | | | | | | |
| HLA- | 0.95-0.98 | 0.41-0.73 | <0.001 | <0.001 | 0.82-0.99 | 3 |
| DQA1*01:01/ | | | | | | |
| DQB1*05:01 | | | | | | |
| Overall | 0.83-1.00 | 0.41-1.00 | | | 0.77-1.00 | 27 |

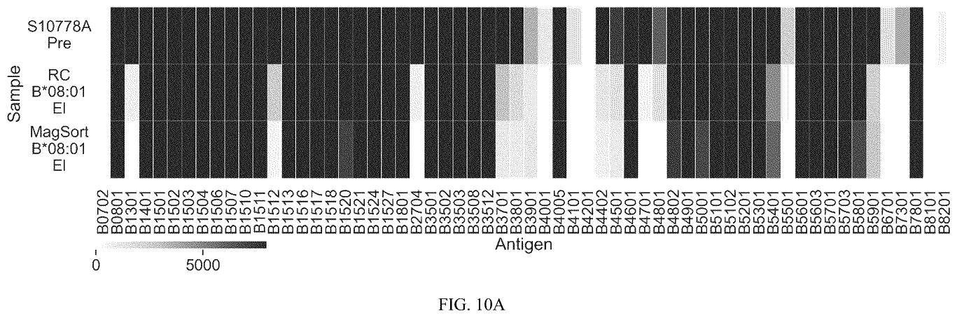

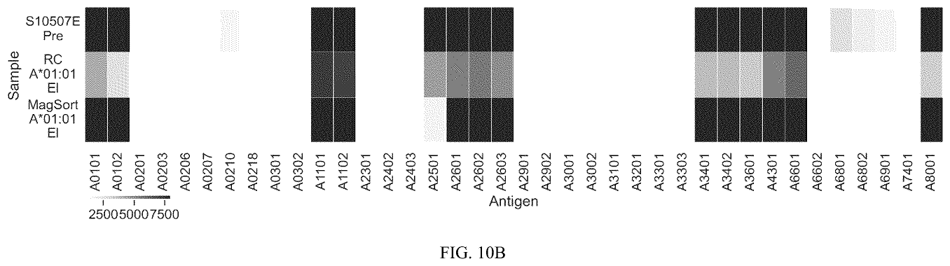

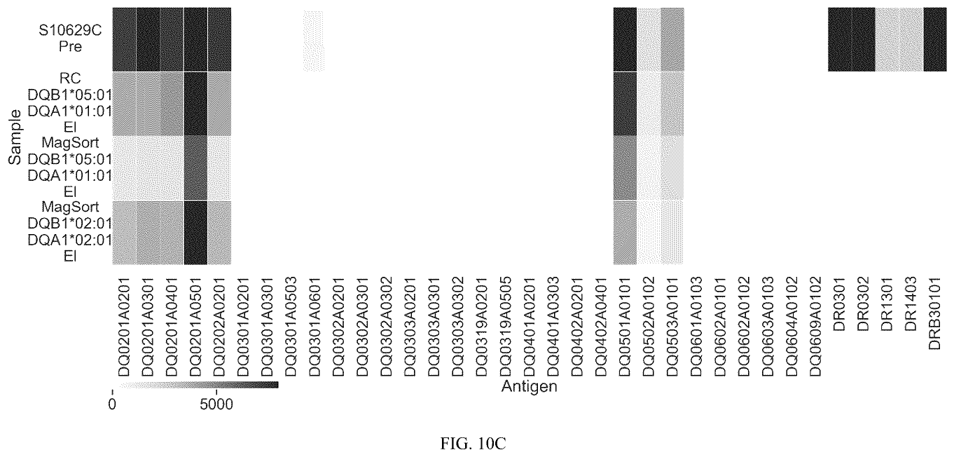

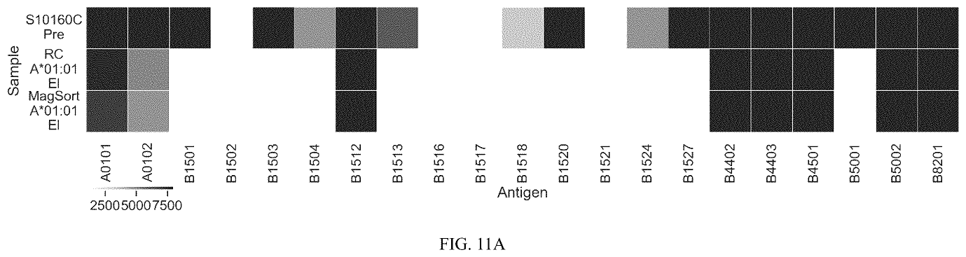

MFI signals of isolated HLA-specific antibodies were compared between the cell method and the magnetic bead method (Table 9 and FIG. 10). Out of the 27 tested sera, 18 show comparable MFI signals with both methods. In 8 sera, MFI signals are higher with the magnetic bead method than with the cell method. For 1 serum, MFI signals are higher with the cell method compared to the magnetic bead method.

Table 9: The comparison of MFI signals for isolated HLA-specific antibodies between the cell method and the magnetic bead method of the disclosure. Column 1: HLA variants. Column 2: number of instances where MFI signals are comparable between the magnetic bead method and the cell method. Column 3: number of instances where MFI signals are greater for the magnetic bead method compared to the cell method. Column 4: number of instances where MFI signals are greater for the cell method compared to the magnetic bead method.

MFI signals comparison between cell
method and magnetic bead method

| HLA variant | Bead = Cell | Bead > Cell | Bead < Cell |
|---|---|---|---|
| HLA-A*01:01 | 2 | 4 | 0 |
| HLA-B*08:01 | 4 | 1 | 0 |
| HLA-C*12:02 | 2 | 0 | 0 |
| HLA-C*12:03 | 2 | 0 | 0 |
| HLA-C*15:02 | 2 | 0 | 0 |
| HLA-DRA1*01:01/ | 4 | 1 | 0 |
| DRB1*11:01 | | | |
| HLA-DPA1*01:03/ | 1 | 1 | 0 |
| DPB1*02:01 | | | |
| HLA-DQA1*01:01/ | 1 | 1 | 1 |
| DQB1*05:01 | | | |

A representative example where MFI signals are comparable for the two methods is shown for isolated HLA-B*08:01-specific antibodies from serum S10778A (FIG. 10A). A representative example where MFI signals are higher for magnetic bead method compared to the cell method is shown for isolated HLA-A*01:01-specific antibodies from serum S10507E (FIG. 10B). An example where MFI signals are higher for the cell method compared to magnetic bead method is shown for isolated HLA-DQA1*01:01/DQB1*05:01-specific antibodies from serum S10629C. MFI signals for isolated HLA-DQA1*02:01/DQB1*02:01-specific antibodies are shown for comparison (FIG. 10C).

### Eplet Identification Using Isolated HLA-Specific Antibodies Binding Reactivity

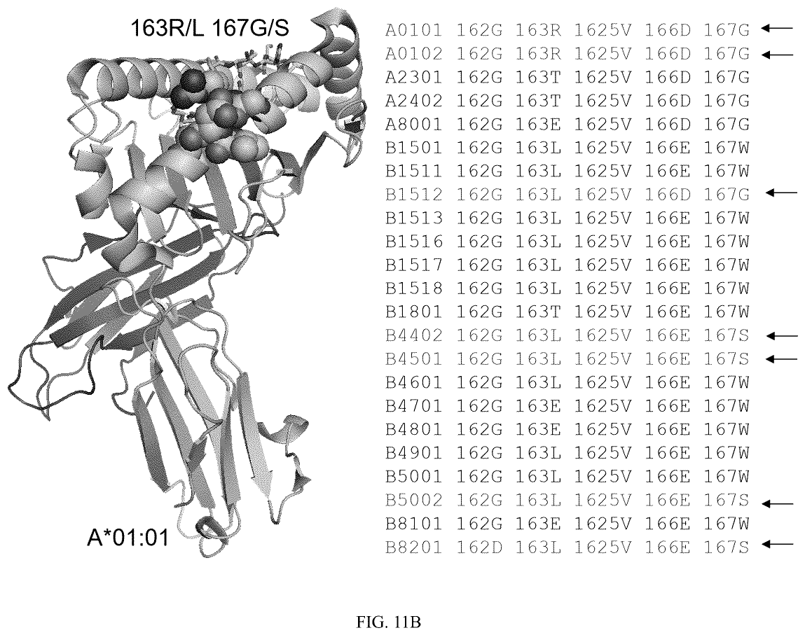

Isolate HLA-A*01:01-specific antibodies from Serum-S10160C binding reactivity on LABScreen™ SAB panel were used for eplet discovery (FIG. 11). Isolated antibodies demonstrate binding reactivity on HLA-A*01:01, A*01:02, B*15:12, B*44:02, B*44:03, B*45:01, B*50:02, and B*82:01. The MFI signals are comparable between the cell method and the magnetic bead method. Multiple sequence alignment was performed using Clustal Omega™. Antigens with positive signals (HLA-A*01:01, HLA-A*01:02. HLA-B*15:12, HLA-B*44:02, HLA-B*44:03, HLA-B*45:01, HLA-B*50:02, HLA-B*82:01) have residue 163 as either arginine (R) or leucine (L) and residue 167 as either glycine (G) or serine (S).

### FCXM Using Isolated HLA-Specific Antibodies

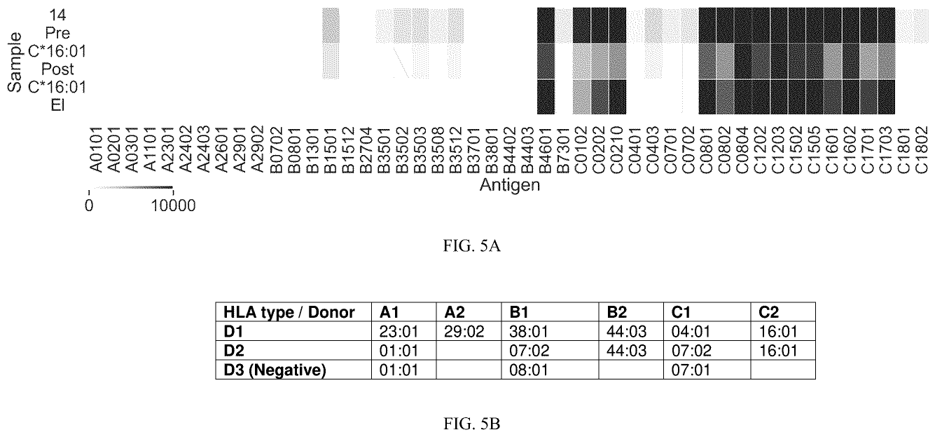

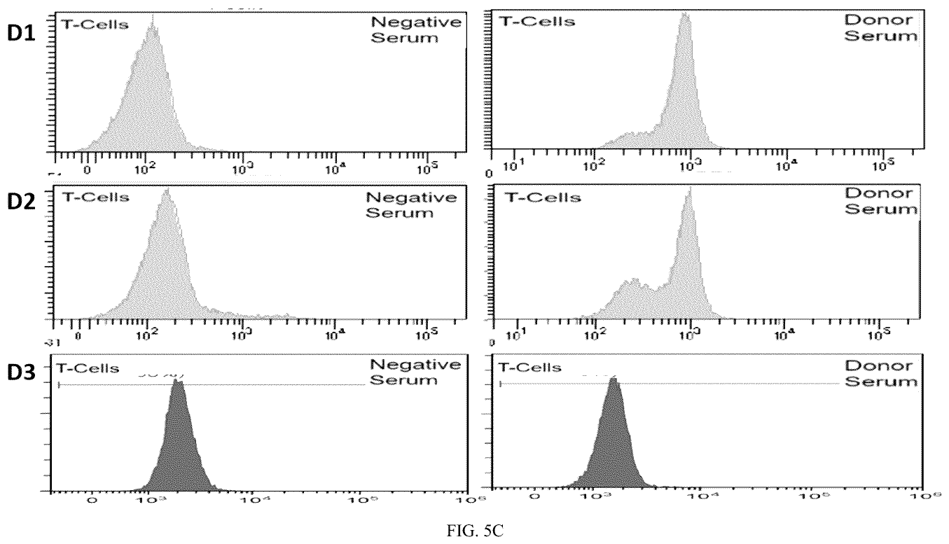

Isolated HLA-specific antibodies were used for FCXM. FIG. 5 shows specific FCXM using donor cells with or without HLA-C*16:01 typing. Serum 14 was used to isolated HLA-C*16:01-specific antibodies. Pre-treatment serum 14's HLA-C*16:01 MFI signal was around 15,000. Isolated HLA-C*16:01-specific antibodies' HLA-C*16:01 MFI signal was around 10,000 (FIG. 5A). FCXM were done using donor D1 (HLA-C*16:01), D2 (HLA-C*16:01), and D3 (Negative) cells. FIG. 5B shows the donors' typing. In FIG. 5C, the ΔMESF of T-cell FCXMs is 2,977 for D1 and 3,219 for D2 for isolated HLA-C*16:01-specific antibodies, indicating a weak positive shift when compared to negative serum. Conversely, negative cells (D3) exhibit no shift between negative serum and isolated antibodies.

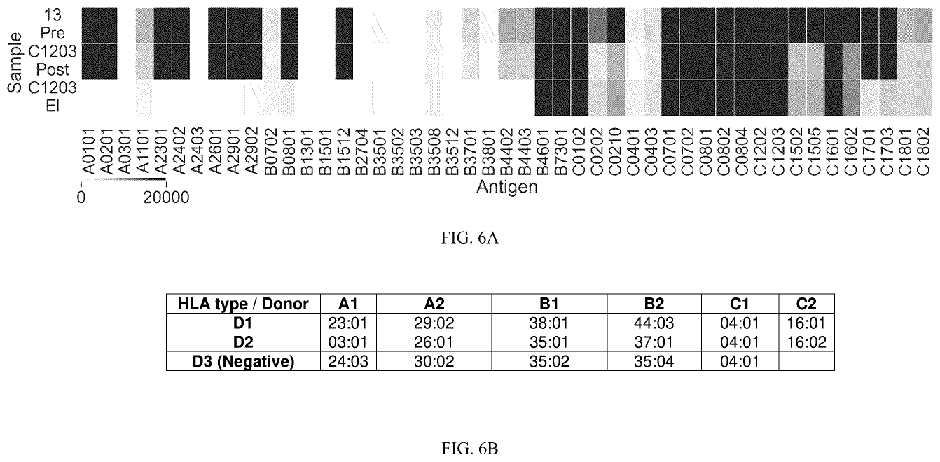

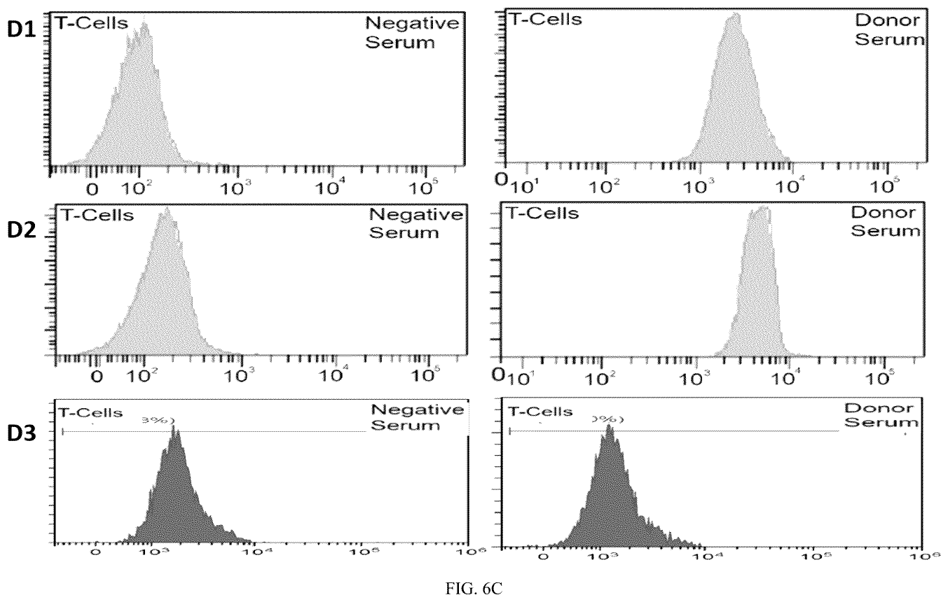

FIG. 6 shows specific FCXM using donor cells with HLA-C*16:01 and HLA-C*16:02 typing. Serum 13 was used to isolated HLA-C*12:03-specific antibodies. Pre-treatment serum 13's HLA-C*16:01 and HLA-C*16:02 MFI signals are around 20,000. Isolated HLA-C*12:03-specific antibodies' HLA-C*16:01 and HLA-C*16:02 MFI signals are around 20,000 and 10,000 respectively (FIG. 6A). Isolated antibodies MFI signals against HLA-A and HLA-B are below 5,000 for most variants. The exception is HLA-B*46:01 and HLA-B*73:01 whose MFI signals are above 10,000. FCXM were done using donor D1 (HLA-C*16:01), D2 (HLA-C*16:02), and D3 (Negative) cells. FIG. 6B shows the donors' typing. In FIG. 6C, ΔMESF of T-cell FCXMs is 9,735 for D1 and 25,819 for D2 cells for isolated HLA-C*12:03-specific antibodies, indicating a positive shift when compared to negative serum. Again, negative cells (D3) exhibit no shift between negative serum and isolated antibodies.

### Discussion

As shown, HLA coated magnetic beads effectively adsorb and elute sera with pre-treatment MFI above 2,500 (1,062 tests). Above this pre-treatment MFI threshold, Elution MFI is expected to range from 30% to 200% of pre-treatment MFI, with higher pre-treatment MFI correlating with higher elution MFI (FIG. 1 and FIG. 7). It's worth noting that, for certain sera, the elution MFI exceeds the pre-treatment MFI, occasionally by a factor of two or higher.

For sera with pre-treatment MFI below 600 (125 sera tested), HLA-specific antibodies elution MFI is expected to fall between 8-19 indicating the absence of HLA-specific antibodies in these sera. Given the low background signal, even when the elution MFI for HLA-specific antibodies is approximately 200, it can still be concluded that HLA-specific antibodies were successfully isolated. An illustrative example is the isolated HLA-A*01:01-specific antibodies from serum S10270C, where the elution MFI is around 200 (FIG. 8). Considering the background signal MFI is around 8, it can be deduced that the HLA binding pattern of HLA-A*01:01-specific antibodies can be parsimoniously explained by eplet 151H, while other eplets are possible.

By employing multiple HLA coated magnetic beads that target distinct HLA variants, diverse HLA-specific antibodies can be isolated from complex sera. FIG. 2 illustrates an instance where serum S10160C was separated into HLA-A*01:01-specific, HLA-A*02:01-specific, HLA-B*13:01-specific, and HLA-C*02:02-specific antibodies, each targeting different eplet sets.

HLA coated magnetic bead isolation reveals that HLA-A*01:01-specific antibodies display a binding pattern not found in the HLA Eplet Registry database. The MFI signal of isolated HLA-A*01:01-specific antibodies is weaker than signals obtained with other tested magnetic beads. However, the elution MFI signal is comparable between the magnetic bead and cell methods (FIG. 11A), indicating that the pattern is not due to a non-natural conformation. The disparity in MFI signal intensity between A*01:01 coated magnetic beads and other variants may be attributed to the specific affinity and/or abundance of these HLA-A*01:01-specific antibodies in serum S10160C. Through multiple sequence alignment, we propose that the isolated HLA-A*01:01-specific antibodies target a new eplet 163R/L167G/S (FIG. 11B).

Similarly, we observe that while HLA-B*13:01-specific antibodies binding pattern can be explained by eplet 80TA, 80TLR, and/or 144QL, unexplained binding patterns towards HLA-B*40, HLA-B*41, HLA-B*45, HLA-B*49, and HLA-B*50 indicate a potential targeting of eplet 45KE.

As mentioned previously, HLA coated magnetic beads facilitate the indirect assessment of both the quantity and binding strength of HLA-specific antibodies. In some cases, HLA coated magnetic beads effectively adsorb and deplete targeted HLA-specific antibodies (FIG. 3). However, in certain sera, potentially due to their high abundance, significant signals for the targeted HLA-specific antibodies persisted in the post-treatment sera even after magnetic bead adsorption (FIG. 4). By diluting these sera to a level where magnetic beads can deplete the HLA-specific antibodies, differences in abundance and strength of HLA-specific antibodies among sera can be comparatively evaluated.

The magnetic bead method demonstrated similarity to the cell method across 27 sera and 8 HLA variants tested. HLA binding patterns were consistent for both methods. Three examples are shown in FIG. 10. For 18/27 sera tested, MFI signals were comparable, while for 8/27 sera tested, the magnetic bead method exhibited higher MFI signals. Notably, increased MFI signals for the magnetic bead method did not correlate with elevated background signals. HLA coated magnetic beads, with estimated higher HLA molecule density than cells, required only around 400,000 beads compared to around 10,000,000 cells for equivalent signals. Instances of higher magnetic bead MFI signals (2 to 5-fold) align with the density difference, suggesting enhanced HLA-specific antibody capture efficiency due to increased HLA molecule density on magnetic beads.

In the case of serum S10629C where the MFI signals for isolated HLA-DQA1*01:01/DQB1*05:01-specific antibodies are lower for the magnetic bead method compared to the cell method, it's possible that the HLA-DQA1*01:01/DQB1*05:01 antigen on the magnetic beads might not be optimal for isolating that antibody. However, using another set of magnetic beads targeting HLA-DQA1*02:01/DQB1*02:02, a similar HLA-specific profile was obtained from serum S10629C. The binding patterns from HLA-DQA1*01:01/DQB1*05:01 coated magnetic beads, HLA-DQA1*01:01/DQB1*05:01 expressed cells, and HLA-DQA1*02:01/DQB1*02:02 coated magnetic beads suggest that the isolated HLA-specific antibodies target eplet 77R. This demonstrates the effectiveness of employing various HLA coated magnetic beads that target distinct HLA variants to identify and verify eplets.

HLA coated magnetic beads can be employed to extract specific signals from sera for flow cytometry crossmatching, enabling the evaluation of their reactivity against specific donor cells. In this study, the inventors have shown that magnetic bead isolation reduces the ambiguity of FCXM results (FIGS. 5 and 6). Healthy cells express HLA-A and HLA-B at more than a 10-fold higher level than HLA-C (see, Apps et al. J Immunol Baltim Md 1950. 2015; 194(8):3594-3600. doi: 10.4049/jimmunol.1403234). Using pre-treatment serum 13 and serum 14, one might argue that positive ΔMESF from FCXM was due to reactions against HLA-A*01:01, HLA-A*03:01, HLA-A*23:01, HLA-A*26:01, HLA-A*29:01, HLA-A*29:02, HLA-B*07:02, HLA-B*35:01, HLA-B*37:01, HLA-B*38:01, HLA-B*44:03, HLA-C*04:01, or HLA-C*07:02 on donors' cells. However, isolated antibodies using HLA coated magnetic beads of the disclosure do not react against these HLA variants, indicating that positive ΔMESF on donors' cells resulted from the binding of the isolated antibodies to HLA-C*16:01 or HLA-C*16:02 on donors' cells. In other words, post magnetic bead treated serum is mono-specific to HLA-C*16:01 or HLA-C*16:02 alleles on donor cell for their serological equivalence study by FCXM.

Recent studies have demonstrated that prospective allocation of donor organs based on eplet-level matching and mismatching improves graft survival. HLA coated magnetic beads of the disclosure facilitate the isolation of specific HLA DSAs from complex sera, enabling further identification and in-depth studies. Additionally, the magnetic bead method described herein proves useful for its simplicity and applicability in cases where cells are unavailable. In conclusion, HLA coated magnetic beads of the disclosure, and the methodology of use thereof, represent a valuable tool for the HLA community, contributing to eplet-based matching strategies in patient-donor selections.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, methods, uses, compositions and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, processes, products, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the systems, methods, uses, compositions and/or products disclosed herein may be made without departing from the scope of the disclosure or of the invention, which is defined in the appended claims. All changes which come within the meaning and range of equivalency of the claims are to be embraced within their scope.

1. A method comprising:
a) contacting a composition comprising a magnetic bead having a human leukocyte antigen (HLA), or extracellular domain thereof, immobilized on the magnetic bead, with a liquid sample to specifically bind antibodies present in the liquid sample to HLA immobilized on magnetic beads;
b) separating magnetic beads having antibodies bound to HLA of the magnetic beads from the liquid sample; and
c) isolating the antibodies of b).

2. The method of claim 1, wherein the HLA is selected from those set forth in Tables 1-6.

3. The method of claim 1, wherein the composition comprises a plurality of magnetic beads, and wherein each magnetic bead of the plurality has a different immobilized HLA.

4. The method of claim 3, wherein the different immobilized HLA comprise 2 or more of those set forth in Tables 1-6.

5. The method of claim 4, wherein the different immobilized HLA comprise at least 10, 20, 30, 40, 50 or more of those set forth in Tables 1-6.

6. The method of claim 4, wherein the different immobilized HLA comprise at least 5, 6, 7 or 8 of those set forth in each of Tables 1-6.

7-10. (canceled)

11. The method of claim 4, wherein the different immobilized HLA consist of those set forth in Tables 1-6.

12-16. (canceled)

17. The method of claim 1, wherein the liquid sample is blood.

18. The method of claim 1, wherein the liquid sample is serum.

19. The method of claim 1, wherein the antibodies are donor specific antibodies.

20. The method of claim 1, further comprising performing one or more of epitope analysis, crossmatching analysis, or antibody binding kinetic analysis.

21-35. (canceled)

36. A composition comprising:
a magnetic bead having a human leukocyte antigen (HLA), or extracellular domain thereof, immobilized on the magnetic bead.

37. The composition of claim 36, wherein the HLA is selected from those set forth in Tables 1-6.

38. The composition of claim 36, wherein the composition comprises a plurality of magnetic beads, and wherein each magnetic bead of the plurality has a different immobilized HLA.

39. The composition of claim 38, wherein the different immobilized HLA comprise 2 or more of those set forth in Tables 1-6.

40. The composition of claim 39, wherein the different immobilized HLA comprise at least 10, 20, 30, 40, 50 or more of those set forth in Tables 1-6.

41. The composition of claim 39, wherein the different immobilized HLA comprise at least 5, 6, 7 or 8 of those set forth in each of Tables 1-6.

42. (canceled)

43. The composition of claim 41, wherein the different immobilized HLA comprise at least 5, 6, 7 or 8 of those set forth in Table 1, at least 5, 6, 7 or 8 of those set forth in Table 2, at least 5, 6, 7 or 8 of those set forth in Table 3, at least 5, 6, 7 or 8 of those set forth in Table 4, at least 5, 6, 7 or 8 of those set forth in Table 5, or at least 5, 6, 7 or 8 of those set forth in Table 6.

44. (canceled)

45. The composition of claim 39, wherein the different immobilized HLA comprise those set forth in Tables 1-6.

46. The composition of claim 39, wherein the different immobilized HLA consist of those set forth in Tables 1-6.

47-50. (canceled)
